(19) United States
(12) Patent Application Publication
(10) Pub. No.: US 2026/0216283 A1
(43) Pub. Date: Jul. 30, 2026

(54) HOXA3 TREATMENT METHODS FOR ACCELERATING WOUND HEALING IN AGED NON-DIABETIC MICE

(71) Applicant: SHIP OF THESEUS LLC, Eagleville, PA (US)

(72) Inventor: Jeremy Elser, Eagleville, PA (US)

(21) Appl. No.: 19/144,325

(22) PCT Filed: Dec. 27, 2023
(86) PCT No.: PCT/US2023/085993

§ 371 Date: Jun. 27, 2025

Related U.S. Application Data
(60) Provisional application No. 63/477,354, filed on Dec. 27, 2022.

(51) Int. Cl. A61K 38/17; A61K 47/38; A61K 48/00; A61P 17/02

(52) U.S. Cl. CPC A61K 38/1709; A61K 47/38; A61K 48/005; A61P 17/02

(57) ABSTRACT

Chronic wounds are characterized by a persistent, hyper-inflammatory environment that prevents progression to regenerative wound closure. Such chronic wounds are especially common in diabetic patients, often requiring distal limb amputation, but occur in non-diabetic, elderly patients as well. Induced expression of HoxA3, a member of the Homeobox family of body patterning and master regulatory transcription factors, has been shown to accelerate wound closure in diabetic mice when applied topically as a plasmid encased in a hydrogel. We now provide independent replication of those foundational in vivo diabetic wound closure studies and expand upon them with minimal dose threshold estimation. Furthermore, we observed similarities in natural wound healing velocity between aged non-diabetic mice and young diabetic mice, which provided motivation to test topical HoxA3 plasmid in aged non-diabetic mice, where we again observed accelerated wound healing. We did not observe any gross adverse effects macroscopically or via local histology in these short studies. Whether as a plasmid or future alternative modality, topical HoxA3 is an attractive translational candidate for chronic wounds.

### CROSS-REFERENCE TO RELATED APPLICATIONS

This application claims benefit of priority pursuant to 35 U.S.C. § 119 (e) of U.S. provisional patent application No. 63/477,354 entitled “HOXA3 Treatment Methods for Accelerating Wound Healing Aged Non-Diabetic Mice,” filed on 27 Dec. 2022, which is hereby incorporated by reference in its entirety.

### FIELD

The disclosed processes, methods, and systems are directed to compositions, methods, and systems for accelerated wound healing in subjects with compromised wound healing, for one example aged subjects.

### SEQUENCE LISTING

The instant application contains a Sequence Listing which has been submitted electronically and is hereby incorporated by reference in its entirety.

### BACKGROUND

Various patient populations suffer from impaired wound healing, which may result in complications. In some cases, patients with wounds, from these populations, may require significantly longer recovery times, for example from weeks to months, increasing the risk and severity of complications. In some cases, these patient populations include aged patients, and patient with diabetes.

Treatments for diabetic foot ulcers (DFUs) span many modalities, yet efficacy is difficult to assess due to small study sizes and a lack of head-to-head comparisons. As evidenced by the existing progression to amputation rate, DFU treatment is still insufficient. The primary therapies still revolve around basic wound care, such as cleaning, debridement, antiseptics, and bandaging. Dressings comprised of biological materials such as human placental tissue, human amnion and chorion, umbilical cord membranes, and even bovine collagen and shark cartilage are commercially available and generally achieve 30-70% closure at 12 weeks whereas controls range from 20-30%. Though one amniotic tissue graft marketed as Epifix did close 31 out of 32 wounds at 12 weeks, where as controls closed only 18 of 35 in a 2016 clinical trial. These treatments require human or animal tissue. Biologically-defined active treatments include rhEGF or PDGF growth factors embedded in topical patches, but the results merely halve the 12 week failure rate, akin to most human tissue grafts.

Normal wound healing progresses through an overlapping sequence of phases starting with clotting, followed by inflammation initiated by neutrophils and macrophages to control introduced pathogens, re-epithelialization by keratinocytes, subepithelial angiogenesis, wound contracture, and scar remodeling of interim extracellular matrix. Multiple factors contribute to impaired wound healing in DFUs, including decreased growth factor production, angiogenesis, macrophage regenerative function, and keratinocyte and fibroblast migration and proliferation. Specifically, the milieu of wounds must transfer from an initial inflammatory phase to a regenerative phase for healing to progress. However, etiology of wound-repair defects in different patient populations is believed to vary between populations, making it unlikely that a single therapy could successfully treat multiple populations.

What is needed are therapeutic compositions and methods for effectively treating and/or aiding wound healing, especially among at-risk populations.

### SUMMARY

Disclosed herein are compositions, methods, and systems for enhancing wound healing in a patient in need thereof. In one aspect, a method of enhancing wound repair in a patient is disclosed, the method comprising identifying a wound in a patient, applying a composition comprising HOXA3 to the wound, allowing at least one cell or tissue to be contacted with the HOXA3 and allowing the wound to heal. In many embodiments, the patient may be aged and/or human. In many embodiments, the HOXA3 is a coding sequence, for example an mRNA coding for HOXA3, and/or the composition may include methylcellulose. The method may further include a step of re-applying HOXA3 after the applying step.

In another aspect, a system for enhancing wound repair in a patient is disclosed, the system comprising an applicator, and a composition compromising HOXA3. In some embodiments, the HOXA3 is a coding sequence, for example an mRNA coding for HOXA3. In some embodiments, the applicator is solid or semi-solid, and may include methylcellulose and/or may be a patch configured to contact the wound.

### BRIEF DESCRIPTION OF THE DRAWINGS

FIG. 1 depicts wound measurement procedure. Wounds were measured manually using digital calipers by taking 4 measurements and averaging. The first measurement was aligned head-to-tail and the subsequent measurements were aligned at 45 degrees to each previous measurement to obtain even coverage. The final average was recorded as the size for the given wound for the given day.

FIG. 2 shows HOXA3 accelerates closure of diabetic wounds. (A) The negative control (Green Fluorescent Protein, GFP) treated wound size was measured as 76% of original wound size, while the HoxA3 treated was measured at 63% of open wound 9 days after wound formation. Percentage closure is defined as measured diameter divided by initial wound diameter (i.e. 6 mm) (B) Representative images of wounds 9 days after wound induction. HoxA3 mice were generally re-epithelized giving the appearance of clearer skin vs the scabbed control. Statistical significance determined as p<0.05 analyzed by Mixed Effects ANOVA.

FIG. 3 shows Histopathology scoring of 9-day diabetic wound healing Fibroblast Cell Density, Granulation Tissue Formation, and Percent of Cohort Fully Epithelialized (which is a global count and thus has no error bars) were statistically significant in favor of HoxA3. Definitions for histopathological scoring criteria are listed in the methods section.

FIG. 4 Representative Histology in 9 day study of HoxA3 plasmid patches in diabetic mouse wounds. (A) Example H&E wound stain highlighting newly formed blood vessels (black arrows). (B) Fibroblast cell density near wound site. (C) Epithelialization of wound site. In GFP treated red bracket shows open lesion, while black line highlights infiltration of immune debris. In the HOXA3 treated, blue line highlights completely healed wound. Healthy tissue is highlight by black dotted lines, and healthy hair follicles are noted by black arrows.

FIG. 5 shows HOXA3 variable dosing and variable dose frequency studies in diabetic mice. (A) Male mice, n=5, were wounded as described and treated with wafers that were replaced ranging from three times, once, and non-treatment control. Treatments were applied weekly from time of wound induction. As a superiority was found for the groups treated three times, a second study was conducted to vary plasmid content using this protocol. (B) In this study, male mice (n=5) were wounded and treated with either 25 μg or lower dose treatments replaced weekly over three weeks (i.e. 3 times total, which was the superior treatment from the variable-frequency study). Data represent mean values; error bars represent S.E.M.

FIG. 6 shows results from mice wounded and observed for 7 days. Cohorts included aged wildtype mice (18 months), young wildtype mice 12 weeks or young db/db mice (12 weeks). Young mice recovered roughly 80% (i.e. measured wound diameters were 80% lower than initial wound size of 6 mm), while aged and db/db mice recovered only about 20% by day 7. (B) column analysis of the final day of wound measurement. ns non-significant, ****p<0.0001, statistics determined by One-Way ANNOVA. Data represent mean values; error bars represent S.E.M.

FIG. 7 presents results from studies showing that HoxA3 accelerates wound healing in aged, nondiabetic mice. Male (n=10) and female (n=10) aged (18 months) mice were wounded by dual punch. Panel A shows results from wound size was measured weekly. Wounds were healed by 3 weeks. Panel B shows HoxA3 mice were significantly more healed at 2 weeks for both genders. Panel C shows blood glucose measured at week 3 was ~180 mg/dL. For all panels, ns non-significant, ****p<0.0001, statistics determined by One-Way ANNOVA. Data represent mean values; error bars represent S.E.M

FIG. 8 presents the genetic sequence of one embodiment of human wildtype HoxA3 (Accession No. NM_030661 and 043365; see uniprot.org/uniprot/043365; SEQ ID NO: 1) used in the plasmid studies (with amino terminal translated peptide sequence of unexpressed genetic sequence SEQ ID NO:4), with an inserted Myc tag and His tag at carboxy-terminus (SEQ ID NO:5).

FIG. 9 shows time-course line graph of an in vitro scratch assay in which a layer of human Dermal Microvascular Endothelial Cells (DMECs) fill in an artificially created scratch in the monolayer over time. The bar graph highlights scratch fraction remaining open at 36 hours under various treatment protocols indicating that incubation with human recombinant HoxA3 protein (with either TAT or PenSV40 cell penetrating peptide motifs prepended to the HoxA3 protein molecules) accelerates closure.

FIG. 10 shows bar graph of oil deposits per cell for various treatments and micrographs of treated cell populations from the Raw 246.7 human macrophage line after co-incubation with oxidized low density lipoprotein (Ox-LDL). The histogram highlights that co-culture with human recombinant HoxA3 protein (with either TAT or PenSV40 cell penetrating peptide motifs prepended to the HoxA3 protein molecules) prevent uptake of Ox-LDL.

FIG. 11 graph showing HoxA3 (m), a recombinant protein derived from a modified sequence of wildtype human HoxA3, closes diabetic wounds faster than HOXA3 (wt). Panel A shows average wound closure trajectory in a diabetic mouse model of skin wound; Panel B shows average AUC of wounds over trajectory.

FIG. 12 shows amino acid sequence alignments of HoxA3 vs HoxB3 (Panel A; SEQ ID NO: 1 vs SEQ ID NO:2) and HoxA3 vs BoxD3 (Panel B; SEQ ID NO:1 vs SEQ ID NO: 3). Also shown are percent Identities (49 and 60% respectively) and Positives (59 and 70%, respectively). Sequences: HoxA3, see uniprot. org/uniprot/043365; HoxB3 see uniprot.org/uniprot/P14651; and HoxD3 see uniprot.org/uniprot/P31249.

FIG. 13 shows protein manufacturing protocol for TAT-HOXA3 (m) and TAT-HOXA3 (wt).

FIG. 14 shows purification and validation of protein isolation (TAT-HOXA3 (wt) shown).

FIG. 15 shows results from scratch defect assay on primary hDMECs (top) and primary keratinocytes (bottom) from a 55 year old patient.

FIG. 16 shows results from primary human monocyte polarization assay.

### DETAILED DESCRIPTION

Disclosed herein are compositions, methods, and systems for treatment of impaired wound healing in patients suffering from same. In many embodiments, the treatments may involve applying a composition comprising HOXA3 to a wound. In many embodiments, the HOXA3 may be applied as a protein or a nucleic acid coding sequence, for example a DNA plasmid or an mRNA sequence. The disclosed HOXA3 may be wild-type HOXA3 or a modified HOXA3, in one example a mammalian HOXA3. Modified HOXA3 may comprise one or more amino acid deletions, substitutions, and/or additions, or the HOXA3 may include one or more moieties, for example moieties that may add functionality to the HOXA3. The disclosed moieties may aid HOXA3 cell targeting, stability, degradation resistance, etc. In one embodiment, the modification is a cell penetrating peptide or CPP. In some embodiments, the HOXA3 composition is applied via a solid or semisolid support, for example a wafer or patch that may be impregnated with the HOXA3 and/or the HOXA3 may be added or re-added after the support is applied to the wound. In many embodiments, the disclosed compositions, methods, and systems may enhance healing of the wound compared to untreated wounds.

Various patient populations have impaired wound healing. Current treatments that accelerate wound healing, and are applicable to different patient populations, include debridement and hyperbaric oxygen therapy. However, these treatments require specialized equipment and trained staff and may require long hospitalizations. Meanwhile, there are topical treatments for at-home use like wound dressings. Most of these deliver antibacterial agents directly at the site of infection and reduce bacterial load to promote wound protection and healing. However, studies are insufficient in demonstrating actual efficacy at accelerating wound healing. Furthermore, multiple mechanisms contribute to wound healing and such treatments treat the symptoms, not the underlying mechanisms of chronic wounds. Some treatments, like EPIFIX and REGRANEX, are based on growth factors key to wound healing (i.e. recombinant platelet-derived growth factor; PDGF). However, these treatments are costly and recombinant proteins like REGRANEX do not persist in harsh protease-rich chronic wound environment. Furthermore, although REGRANEX is the only FDA-approved growth factor treatment for impaired wound healing (in diabetic patients), its use has been limited due to high cost and adverse side effects including possible increased risk of malignancy. Taken together, there is a need for a treatment that can target the multifactorial problem of impaired wound healing.

Diabetic foot ulcers (DFUs) are a common and devastating complication of diabetes that may take weeks to several months to heal due to impaired wound healing. As of 2018, 10.5% of the US population has diabetes. Between 1-4% of the U.S. diabetic population experiences a DFU in a given year, a statistic that has not changed in the past two decades. Among DFUs, 14-24% will require lower limb amputation. Therefore, about 80,000 US DFU amputations per year occur in the U.S. In addition to the decreased quality of life, per amputation hospitalization costs are $12-16k in the US.

Homeobox protein A3 (HOXA3) is a transcription factor that promotes diabetic wound healing. Mace et al delivered HOXA3 expression plasmids to wounds in young Leprdb/Leprdb (db/db) diabetic mice using the methylcellulose topical gene delivery patch. The application of HOXA3 expression plasmids significantly improved wound closure rates in db/db mice compared to vehicle plasmid as negative control. In a large diameter wound model (2.5 cm), HOXA3 treatment improved wound closure rates detectable as early as 7 days, and incited closure of all wounds by 42 days versus 77 for untreated mice. Further rigorous in vitro and in vivo studies demonstrated enhanced neovascularization, keratinocyte migration, and endothelial cell invasion and migration compared to negative vehicle control. The mechanisms underlying the wound healing ability of HOXA3 has also been preliminarily investigated. Al Sadoun et al. showed that macrophages transduced with HOXA3 had inhibited M1 polarization and promoted M2 polarization via regulation of Pu. 1/Spi1 and Stat6 compared to negative control mCherry transduction. Furthermore, Mace et al. demonstrated HOXA3 treatment mobilized and recruited endothelial progenitor cells while attenuating inflammatory pathways when compared to control mice and Mahdipour et al. demonstrated HOXA3 promotes the differentiation of hematopoietic progenitor cells into proangiogenic Gr-1+CD11b+ myeloid cells that stimulate neovascularization. Together, these published in vitro and in vivo data provide evidence that HOXA3 may affect angiogenesis, re-epithelialization, and pro-healing macrophage polarization.

Wildtype aged mice appeared to respond to injury with impaired healing. However, etiology for this impairment has not been fully described. Applicants, show, herein that HoxA3 is an effective wound healing treatment for aged mammals.

While the ability of HoxA3 plasmid (or paralogs like HoxB3, HoxD3, etc.) to accelerate diabetic wound closure in vivo had been shown previously by the Mace and Boudreau labs, one of skill in the art would not reasonably expect that HOXA3-based therapy would have a similar effectiveness in non-diabetic aged mice. Applicant's ability to treat wounds in aged mice is unexpected. HoxA3 was shown previously in vitro to favor macrophage polarization to M2 gene expression, stimulate endothelial cell motility, and stimulate keratinocyte motility.

Applicant hypothesized that these factors may also be affected during healthy aging, which may be referred to as “inflammaging.” However, given that high blood glucose itself is known to be deleterious to wound healing via formation of Advanced Glycation Endproducts (AGEs) which are not directly targeted by HoxA3, the effectiveness of HoxA3 in non-diabetic aging wounds was unclear.

Chronic wounds, both diabetic and nondiabetic, are a source of significant morbidity and cost affecting 1-2% of the general population in developed countries. Among the elderly (age 65+), prevalence for chronic ulcers, pressure ulcers, and DFUs are 2.3%, 1.8%, and 0.7%, respectively. The extremely elderly and ill patients are most at risk, and nursing home patients are particularly vulnerable to pressure wounds with a prevalence of 2.5%. Despite the severity of this problem, no new treatments have been approved for chronic wounds since 1997 (becaplermin gel).

The wounds in this study were inflicted as sterile wounds to achieve controlled, homogenous injuries. While clinical reports vary, roughly 70% of chronic wounds are observed to have microbial biofilm presence. Infections may need to be resolved prior to treatment with HoxA3, but this is untested. Wounds in the present study were acute, but benefitted from HOXA3 treatments. Thus, in one embodiment, the disclosed HoxA3 therapeutic compositions may be applied by the patient (or other person) at home. Moreover, in many embodiments, the disclosed treatments may be useful for administration to common/everyday wounds in the aged or elderly. In many embodiments, the disclosed compositions and treatments may be useful in early intervention, for example with pressure ulcers in a medical, long-term care, or assisted living facilities.

Applicants note that inflammaging may be a cumulative process and may become more prevalent/noticeable in extreme old age. However, the process occurs continuously and may also be accelerated in patients with other health risk factors. Thus, Applicants hypothesized, HoxA3 treatment might be especially beneficial for treating wounds in elderly patients, while still being effective in middle-age patients that have begun the inflammaging process and/or have other risk factors. Without wishing to be limited by theory, Applicants surmise that, while the use of HoxA3 in inflamed wounds in youthful patients may be less effective (compared to elderly and other at-risk populations), stump wounds, may also be effectively treated with the present compositions and methods. Specifically, stump wounds may become chronic due to abrasion from a prosthetic device, even without microbial involvement, resulting in a hyper-inflammatory environment.

In some embodiments, Applicants use a HOXA3 expressing plasmid to achieve a durable effect on wound healing. In some cases, plasmids may not integrate into the host genome. In other embodiments, HOXA3 protein may be applied to a wound. In many embodiments dosing amounts and schedules may differ based on the type of composition used, due to differences in degradation, cellular uptake, nuclear trafficking, and intracellular half-life.

### Patients

Various patient groups may be effectively treated with the disclosed methods. In some embodiments, the treated patients suffer from chronic or long lasting wounds, for example patients may be selected from aged, diabetic, and amputees. Patients may be located in a medical care setting. In some embodiments, the disclosed patient is at home and may self-administer the disclosed compositions and treatments. In many embodiments, the wound or injury is diabetes-associated, age-associated, chronic, or acute. In many embodiments, the wound or injury may be associated with inflammatory regions, for example an amputation stump wounds.

As defined herein, an ‘aged’ patient may be about 60 years of age, or older, for example about 61 years of age, 62 years of age, 63 years of age, 64 years of age, 65 years of age, 66 years of age, 67 years of age, 68 years of age, 69 years of age, 70 years of age and older.

In some embodiments, the disclosed compositions may be used to treat patients with conditions other than a wound or injury. In one embodiment, the condition may be atherosclerosis, and treatment may aid in preventing foam cell formation. In these embodiments, the disclosed compositions may be administered to a patient systemically and/or included in a coating of a medical device, for example a stent. In stent-based embodiments, HOX may elute from the stent and be taken up by a macrophage at or near a coronary lesion, for example an atherosclerotic plaque. In embodiments, where the administration is systemic, treatment may be more convenient and much of the therapeutic composition can still be expected to reach target cells, especially where the HOX therapeutic composition comprises a liponanoparticle (LNP), as these particles may be preferentially taken up by macrophages.

### Target Cell

Various mammalian cells may be targeted by the disclosed compositions and methods. In most embodiments, the target cells are located at, near, or around a wound or injury, for example within the skin, epithelium, epidermis, dermis, fat layer, blood vessel, etc. In some embodiments, the target cell is one or more of a neutrophil, macrophage, epithelial cell, keratinocyte, and fibroblast.

### Dosing

Various dosing amounts and scheduling are envisioned for treating a wound with the disclosed methods. In many embodiments, wounds may be treated 1-7 times, or more, per week, for example 1× or 3× weekly, or once every 2 weeks. The amount of composition may vary depending upon type of composition and form of HOXA3 (for example, protein, plasmid, and/or mRNA, etc.). In some embodiments, wherein the form of HOXA3 is a plasmid, the amount may vary from about 1 μg to about 25 μg.

### HOX

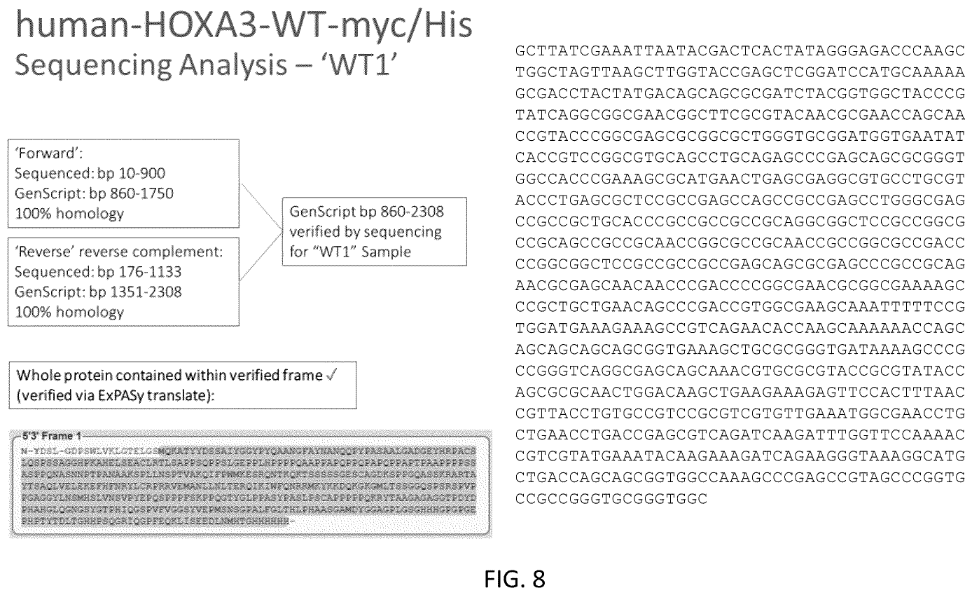

Disclosed herein are various compositions and methods comprising HOX, for example a HOX amino acid sequence that is at least about 45% identical to HoxA3. In some embodiments, the HOX amino acid sequence may be about 80% identical to HoxA3, HoxB3, HoxC3, or HoxD3, which has been shown to affect wound healing in diabetic mice (Hansen, S. et al., Am. J. of Path., Vol. 163, No. 6, 2003). In many embodiments, the HOX is mammalian, for one example, human. In many embodiments, the HOX is HOXA3, for example as presented in FIG. 8. In various embodiments, the HOX may be wild-type/native or non-native/modified HOX. For example, wild-type or modified mammalian HOXA3. Modified HOXA3 may be of various forms. In one embodiment, modified HOXA3 may comprise one or more amino acid deletions, substitutions, and/or additions, or the HOXA3 may include one or more moieties, for example moieties that may add functionality to the HOXA3. In one embodiment, the modified HOXA3 may include one or more substitutions selected from L175D, E176K, and E178K (nomenclature is in reference to HOXB4; HOXA3 nomenclature is L206D, E207K, and E209K). As used herein, HOXA3 (m) may refer to a protein or nucleic acid sequence coding for a protein comprising amino acid substitutions L175D, E176K, and E178K. Disclosed moieties may aid HOXA3 cell targeting, stability, degradation resistance, etc. In one embodiment, the modification is a cell penetrating peptide or CPP to enhance cellular uptake and/or nuclear trafficking of the molecule.

### HOXA3 Composition

The disclosed compositions, methods and systems include a step of contacting at least one mammalian cell or tissue, positioned at or near a wound, with a formulation comprising HOXA3. The formulation may include, without limitation, pharmaceutically acceptable carriers, solids, semisolids, methylcellulose, nanoparticles, poly(lactic-co-glycolic acid) (PLGA) microspheres, lipidoids, lipoplex, liposome, polymers, carbohydrates (including simple sugars), cationic lipids, fibrin gel, fibrin hydrogel, fibrin glue, fibrin sealant, fibrinogen, thrombin, rapidly eliminated lipid nanoparticles (reLNPs) and combinations thereof. In many embodiments, LNPs may aid in targeting target cells, for example macrophages, that may preferentially take up LNPs, as described in Truzzi et al. (In vivo Biodistribution of Respirable Solid Lipid Nanoparticles Surface-Decorated with a Mannose-Based Surfactant: A Promising Tool for Pulmonary Tuberculosis Treatment. Nanomaterials 2020; 10:568-83), which is incorporated by reference in its entirety.

Wounds may be treated with various amounts and various forms of HOXA3. In some embodiments, HoxA3 may be applied as a protein, peptide, or coding sequence. In embodiments where HoxA3 is added as a coding sequence, the coding sequence may be a nucleic acid, for example RNA or DNA. In one embodiment, the coding sequence is RNA, for example mRNA.

The HoxA3 formulation applied to wounds may be in various forms. In many embodiments the formulation is a transcription construct. In some embodiments, the transcription construct may be a nucleic acid sequence coding for HoxA3 or a protein or peptide with at least about 45% identity to HoxA3, for example greater than about 45%, 50%, 60%, 70%, 80%, 90%, or 95%, and less than 100%, 90%, 80%, 70%, 60%, or 50% identity. In some embodiments, the nucleic acid may be an RNA sequence, for example an mRNA sequence. In some embodiments, the transcription construct may be a virus, plasmid or other suitable vector. In one embodiment, the transcription construct may be a plasmid, which may be loaded onto a solid or semi-solid support prior to application to the wound. In one example wafers may be impregnated with the transcription construct, such as a plasmid. In these embodiments, the wafers may be impregnated with 1-100 μg of the transcription construct.

HOXA3 formulations may be delivered to cells and tissues as nucleic acid coding sequences. The coding sequences may be RNA or DNA. Where the coding sequences are DNA, the nucleic acids may be delivered in a lipid nanoparticle composition, viral vector, or plasmid. Where the coding sequences are mRNA, the mRNA may be delivered in RNA-based viral particle or a lipid nanoparticle composition. In some embodiments, the lipid nanoparticles may be created by mixing a solution comprising pre-formed lipid nanoparticles and a solution comprising mRNA to form mRNA lipid nanoparticles.

Messenger RNA, mRNA, is becoming an increasingly useful active ingredient for the treatment of a variety of diseases. mRNA-based therapies involve administration of messenger RNA to a patient in need of the therapy. In most cases, the mRNA is delivered to a patient's cell, where it codes for a protein encoded by the mRNA. Lipid nanoparticles are commonly used to facilitate this delivery. In most cases, the lipid nanoparticle encapsulates the therapeutic mRNA for efficient in vivo delivery of mRNA.

In some embodiments, altering lipid compositions may affect intracellular delivery and/or expression of mRNA coding sequences, to various types of mammalian cells and tissues (e.g., mammalian epithelial cells). In other embodiments, the disclosed particles, lipid nanoparticles, or viral particles, may include one or more sequences, tags, receptors, etc. that aid in targeting the particle to the proper cell, for example epithelial cells.

Disclosed herein are viral vectors comprising one or more nucleic acids coding for HOXA3. Adeno-associated viruses (AAV) are small viruses that infect humans, and belong to the genus Dependoparvovirus (family Parvoviridae). AAV are about 20 nm, replication-defective, nonenveloped viruses, comprising linear single-stranded DNA (ssDNA) genome of approximately 4.8 kilobases (kb). AAV are designed to deliver various genes to cells and tissues. In some embodiments, the AAV includes one or more peroxidase encoding nucleic acid sequences.

### Applicator

An applicator may be used to contain or carry the HOXA3 formulation. In some embodiments, the applicator is a solid or semi-solid. In many embodiments, the disclosed HOXA3 formulation may be added directly to the applicator. In one embodiment, the applicator is a patch or wafer, which may be configured to conform to and/or contact a cell or tissue at or near a wound. In some embodiments, the one or more doses may be applied and/or re-applied directly to the applicator. The disclosed applicator may be, in one embodiment, fabricated, at least in part, from methylcellulose. Alternatively, the disclosed HOXA3 compositions can be incorporated into creams or ointments for use in topical application.

### Enhanced Wound Repair

The disclosed treatment methods may result in enhanced wound repair in patients, compared to similar, untreated wounds, in matched patients. In some embodiments, treated patients may have between 20% and 400% more healing at a timepoint after wounding and/or commencement of treatment. In some embodiments, the timepoint may be from about 1 to about 4 weeks, for one example about 4 weeks. In one embodiment, for example in treated patients the wounds may be more than about 70%, 80%, 90%, or 95% healed compared to wounds in untreated patient that may be about 50% healed at two weeks.

The disclosed treatment methods may result in wounds being healed more quickly than untreated wounds. In some embodiments, the treated wounds may be healed between about a day sooner and 2 weeks sooner, compared to the size of the wound. In one embodiment, a wounds of about 6 mm may heal more than one day, 2 days, 3 days, 4 days, 5 days, 6 days, 7 days, 8 days, 9 days, 10 days, 11 days, 12 days, or 13 days sooner and less than about 2 weeks, 13 days, 12 days, 11 days, 10 days, 9 days, 8 days, 7 days, 6 days, 5 days, 4 days, 3 days, or 2 days sooner than a 6 mm untreated wound.

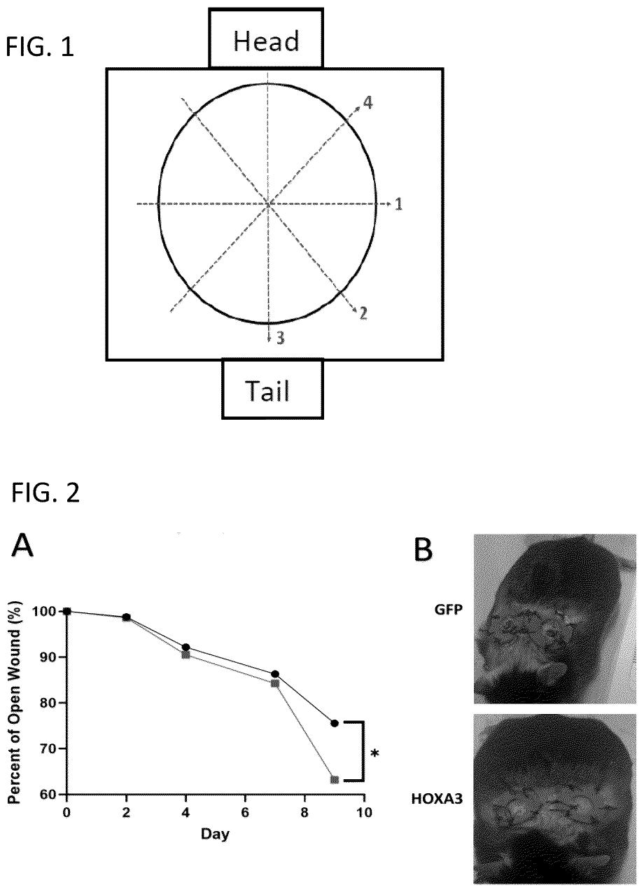

Accelerated wound healing may be measured by various methods. In one embodiment, as disclosed above, measurement may be by macroscopic measuring of various lengths/dimensions of the wound. In these embodiments, the measurement method may be described at FIG. 1. In other cases, measurement may be by microscopic examination. In these embodiments, examination may be of one or more of vascular density, layers of epithelial cells, inflammation, and granulation tissue formation. In various embodiments, more than about 400% more of the treated wounds may be re-epithelialized at a given timepoint compared to untreated wounds. In one example about 80% of treated wounds and about 20% of untreated wounds may be re-epithelialized by the timepoint. As disclosed above, the timepoint may be from about 1 to about 4 weeks

The disclosed treatment methods may be applied in various forms and various application schedules. In some embodiments, the disclosed doses may be applied immediately after wounding, or hours or days after wounding. In many embodiments, doses may be repeated to achieve 1, 2, 3, 4, 5, 6, 7, 8, 9, 10 or more doses. Where the composition is a plasmid applied via an applicator, such as a patch or wafer, the patch or wafer may comprise between about 0.01 and 50 μg, for example 2.0 μg, 1.0 μg, 5 μg, or 25 μg.

### EXAMPLES

### Example 1—HoxA3 Accelerates Wound Healing in Aged Patients

As disclosed below, Applicants identified similarities in the wound healing response of aged-wildtype mice and young diabetic mice. Applicants hypothesized that HoxA3 may be effective in accelerating wound healing, a created a study to test the hypothesis. In these studies, mice were wounded as described previously and treated with 3 doses of 25 μg HoxA3 (treatment applied once at time of wound, and per week after). Both male and female mice were separately tested (n=10). Mice were observed weekly by digital caliper for wound size. All wounds were closed at 21 days. No appreciable difference in wound closure at time of injury or at 1 week post injury. However, at 2 weeks post injury, the treated wounds were consistently healed beyond 90% whereas the untreated wounds were consistently healed only approximately 50%. At 3 weeks, mice were sacrificed and blood glucose was tested, showing no significant difference between the groups. All groups were nondiabetic.

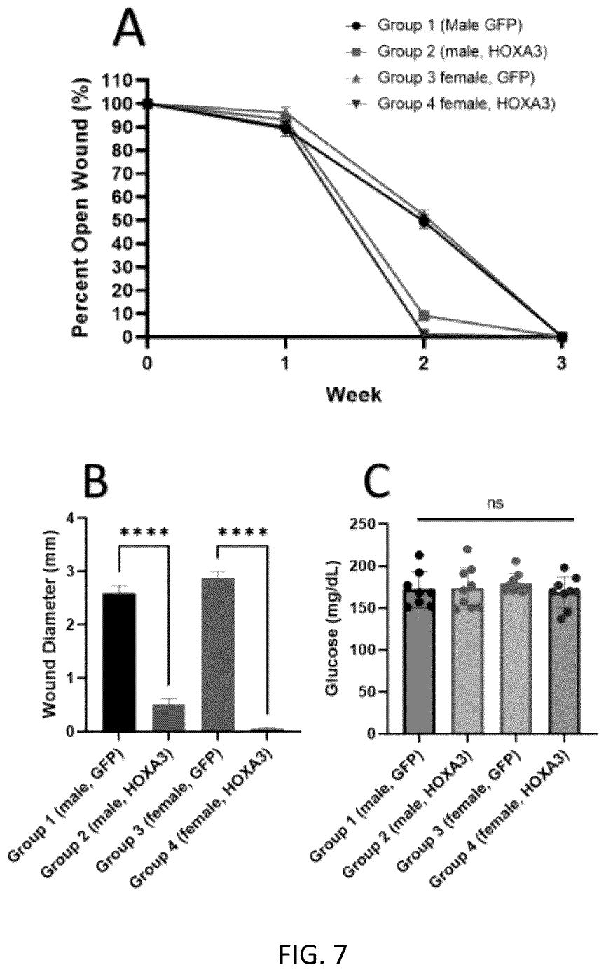

FIG. 7 shows that HoxA3 treatment accelerates wound healing in aged, nondiabetic mice. In these studies, male (n=10) and female (n=10) aged (18 months) mice were wounded by dual punch. Wound size was measured weekly. Wounds were healed by 3 weeks. HoxA3 mice were significantly more healed at 2 weeks for both genders. Blood glucose measured at week 3 was ~180 mg/dL. For all panels, ns non-significant, ****p<0.0001, statistics determined by One-Way ANNOVA. Data represent mean values; error bars represent S.E.M.

### Example 2—Topical HOXA3 Plasmid Accelerates Wound Healing in Diabetic Mice at the Microscopic Levels

Diabetic mice (db/db) were randomized during the acclimation period for punch wounding and subsequent wound size monitoring until Day 10 post-injury, when mice were humanely sacrificed. One animal died during the acclimation period, leaving Group 1 (control) with 9 animals and Group 2 (treatment) with 10 animals. Treatment consisted of GFP (control) or HOXA3 plasmid wafers (25 μg DNA plasmid in 1% methylcellulose, applied as 50 μL patches after 5 hrs of drying on waxed paper) applied to the wounds on Days 0, 2, 4, 7, and 9. Splints and sutures were replaced as needed throughout the study.

The primary intended outcome was histologic, but three representative animals in each group had wounds measured on Days 0, 2, 4, 7, and 9. All animals were weighed on Days 0, 2, 4, 7, and 9. On Day 10, each group was humanely euthanized, and wound areas were collected. From each animal, one wound was fixed in 10% neutral buffered formalin while the other was flash frozen. Fixed wounds were embedded into paraffin blocks for microscopy and blinded pathologist scoring.

At Day 9, the final recorded data point before sacrifice, the HoxA3 cohort rapidly closed from about 85% open to about 65% open, whereas the control group remained at about 80% open. Although Day 9 was the first day of statistical significance, this fact was incidental, as the results were not tabulated until after sacrifice and Day 10 was merely chosen as the halfway point of untreated healing based on prior pilot studies (not shown).

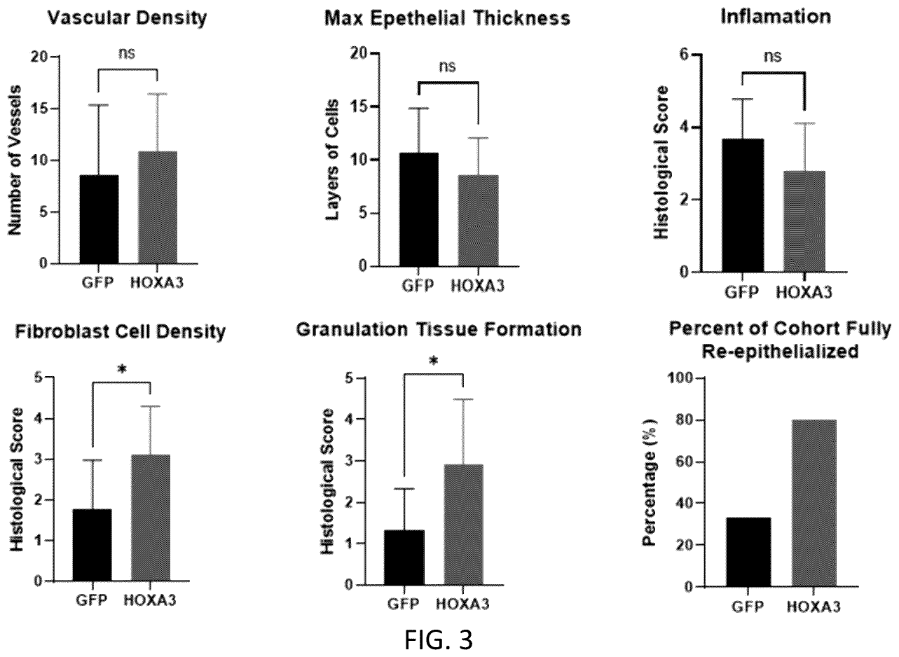

Anonymized wounds were scored by board-certified pathologist analyzed a 400× field from each wound. No statistical difference was noted for Vascular Density, maximum epithelial thickness, or Inflammation; however, HoxA3 dramatically increased granulation tissue formation and nearly 80% of the HoxA3 wounds were totally re-epithelialized compared to only 20% for the controls (FIG. 3).

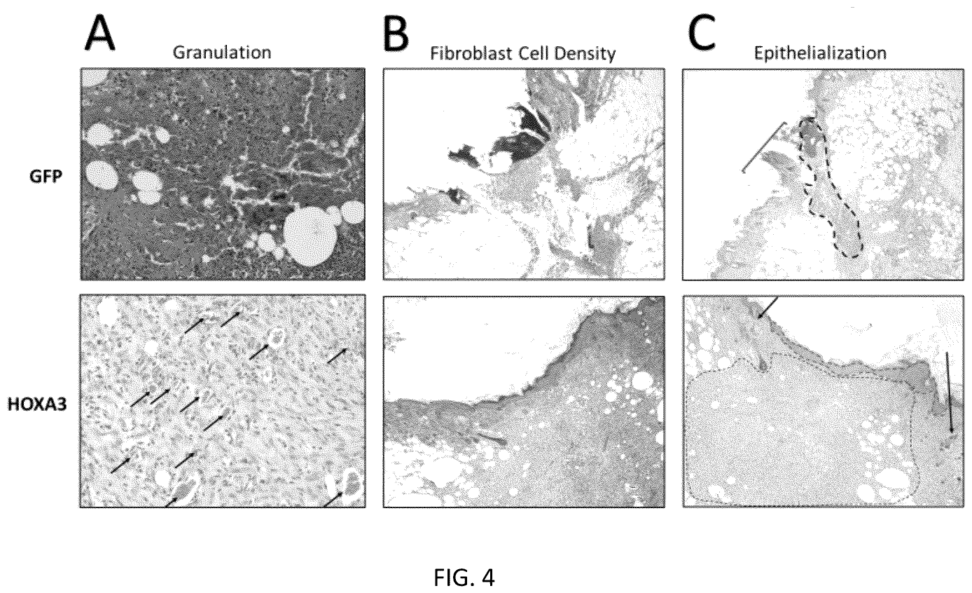

FIG. 3 shows results of histolopathology scoring of 9 day diabetic wound healing, Fibroblast Cell Density, Granulation Tissue Formation, and Percent of Cohort Fully Epithelialized (which is a global count and thus has no error bars) were statistically significant in favor of HoxA3. Also presented are definitions for histopathological scoring criteria in the materials and methods section FIG. 4 top row shows, the annotation marks patent blood vessels with red blood cells (RBCs) and tissue granulation in HoxA3 sample, which are absent in GFP. FIG. 4B second row shows fibroblast cell density marked by the high presence extracellular matrix material. FIG. 4C shows re-epithelialized HoxA3 wound vs open control wound.

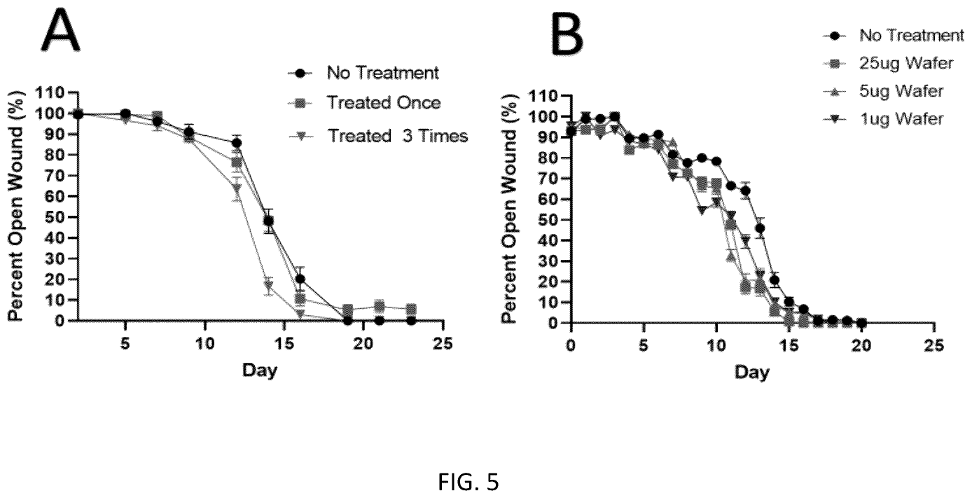

A dose study was conducted to determine a minimum plasmid dosing frequency and volume needed to elicit accelerated healing. The resulting minimum threshold was intended to be used to design additional experiments on improved therapies by determining the optimal dynamic range. First, mice were wounded and treated with 25 μg plasmid patches and monitored every two days for wound size by digital calipers. Mice received either three total doses (1 per week), a single dose, or a non-treated control. Three total dosages (i.e. weekly) was effective while a single dose was not (FIG. 5A).

The impact of dosage quantity was then evaluated by varying plasmid content in the gel patches under the three-dose regime. To improve temporal resolution, observations were made daily. Cohorts included either No Treatment, 25 ug wafer (akin to the most effective dose frequency approach), and intermediate dosages including 0.2 ug, 1 ug, and 5 ug. As shown in FIG. 5B, the 25 ug was nearly completely healed by Day 15 unlike the others.

As shown in FIG. 5B, the 25 ug was nearly completely healed by Day 15 unlike the others. Specifically, Panel A shows results from male mice, n=5, wounded as described and treated with wafers that were replaced ranging from once weekly (3 total), once time at injury, and non-treatment control. A second study was conducted to vary plasmid content using the weekly protocol. In this study, male mice (n=5) were wounded and treated with either 25 ug or lower dose treatments.

### HoxA3 Topical Plasmid Patch Accelerates Wound Healing in Non-Diabetic Aged Mice

Aging is associated with the phenomenon “inflammaging,” as the immune system in older mammals enters an increasingly chronic state of low-grade immune activation with increasing age. Wounds are also known to heal more slowly in elderly human patients than in youth. This effect is known to be partially macrophage mediated and is exacerbated in diabetic patients. In a pilot study assessing the relative wound healing rates of aged wild-type mice young wild-type mice, and young diabetic mice (12 weeks of age), young wild-type mice healed quickly, requiring approximately one week. Whereas, both young db/db and aged wildtype mice were similarly only 20% healed by Day 7. Applicants hypothesized that aged nondiabetic mice may benefit from HoxA3 plasmid due to the similar trajectory for those two populations observed in this pilot experiment.

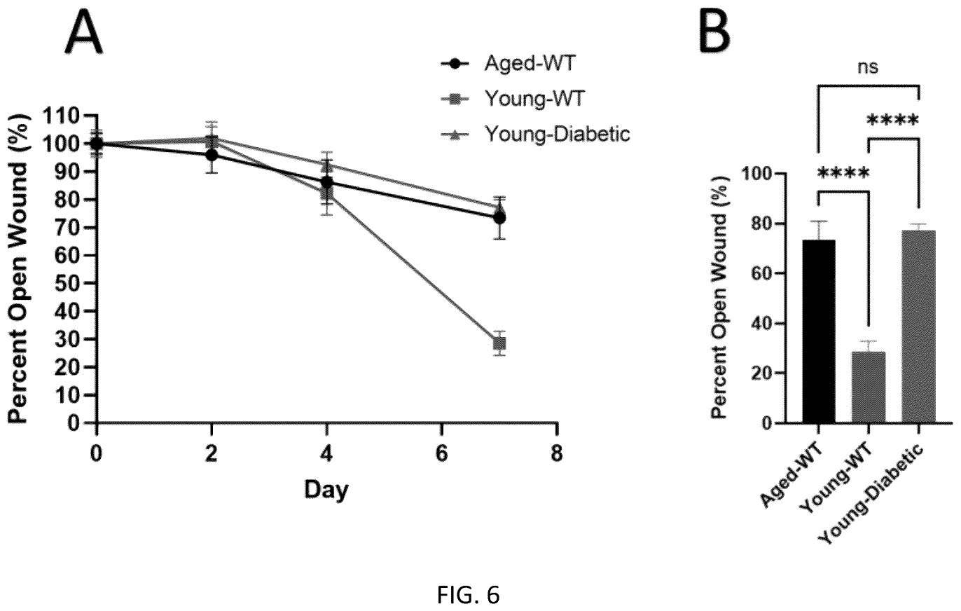

FIG. 6 shows results from mice wounded and observed for 7 days. Cohorts in this study included aged wildtype mice (18 months), young wildtype mice 12 weeks, or young db/db mice (12 weeks). Young mice recovered roughly 80% while aged and db/db mice recovered to only about 20% by day 7. Panel B shows a column plot analysis of the final day of wound measurement. Ns non-significant, ****p<0.0001, statistics determined by One-Way ANNOVA. Data represent mean values; error bars represent S.E.M.

### Example 3—Relative Wound Closures of DMECs Treated with Various HOXA3 Interventions

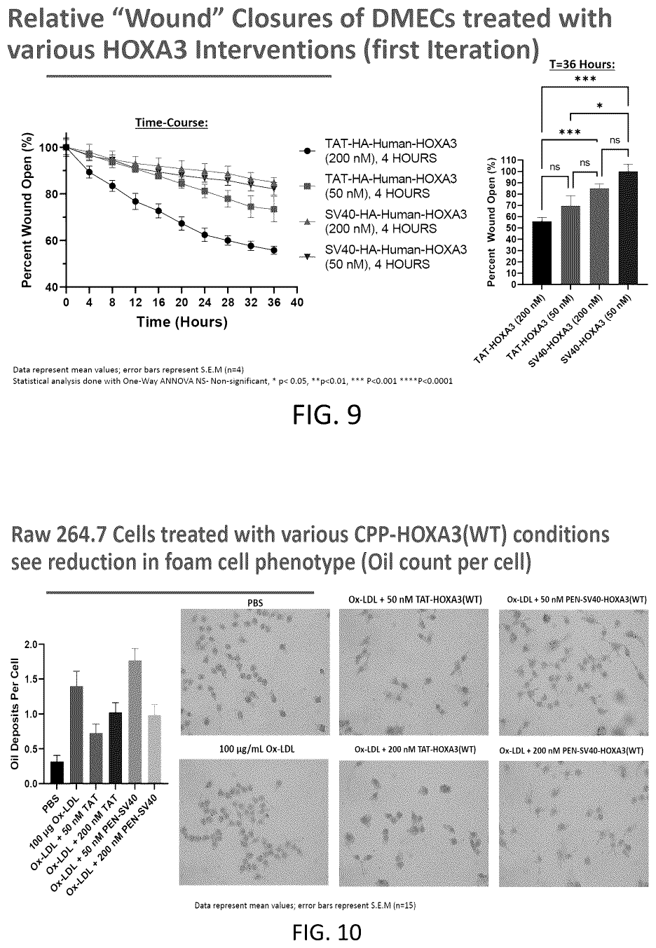

FIG. 9 show results from in-vitro scratch/motility assay comparing HOXA3 fused to a cell penetrating motif of HIV-TAT or Penatrin or no cell penetrating motif. Briefly, scratches were made to disrupt adherent cells and then motility back into the scratch zone was measured as a indication of wound closure. Untreated DMECs were less motile and thus filled the scratch less quickly than DMECs treated with HOXA3 protein. The HoxA3 protein possessed either an HIV-TAT cell penetrating peptide (CPP) motif or a PenatrinSV40 CPP. The TAT was more effective at comparable dosages to the PenatrinSV40. The TAT-HoxA3 displayed dose dependence as the 200 nM dose increased motility beyond the 50 nM dose which itself was superior to the untreated cell closure rate.

Primary Dermal Microvascular Endothelial Cells, “DMEC” cells (ATCC, PCS-110-010) were seeded in a 96-well plate at a density of 12,000 cells/well. Cells were cultured in vendor recommended media overnight to allow cells to adhere. The media was removed and fresh media containing listed HOXA3 treatments (or vehicle control) were supplemented into the growth media for 4 hours. After the 4-hour incubation, the wells were scratched with a P200 pipette tip. The size of wounds over time were measured utilizing the IncuCyte S3 Kinetic Scratch Wound Module.

### Example 4—264.7 Macrophage Cells Treated with CPP-HOXA3 (WT) Show Reduction in Foam Cells Phenotype

FIG. 10 shows studies of foam cell formation in HOXA3 treated and untreated macrophages. Briefly, Raw 264.7 cells, a macrophage lineage, were treated with 100 μg/mL oxidized low-density lipoprotein at the same time as listed HOXA3 treatments (or vehicle control). Cells were cultured with lipids and HOXA3 for a total of 48 hours in DMEM. Once the 4-hour incubation was complete, the media was removed from the cells, which were then washed with PBD prior to staining with Oil Red O.

Untreated macrophages accumulated oil deposits indicative of a Foam Cell (FC) phenotype. Co-culture with HoxA3 protein, either with an HIV-TAT cell penetrating peptide (CPP) motif or Penatrin-SV40 CPP motif, mitigated the formation of oil deposits and caused cells to retain the morphology of non-OxLDL treated cells. The effect was dose dependent, as 200 nM of TAT-HoxA3 was superior to 50 nM. HIV-TAT was more effective per given dose than Penatrin-SV40. However, the 200 nM dose of PenSV40-HoxA3 was also effective. These results indicate that HoxA3 protein exposure prevents macrophages from becoming foam cells in response to treatment with Oxidized LDL.

Oil Red O working solution was made by reconstituting Oil Red O powder (Sigma-Aldrich Cat #MAK194) at a concentration of 30 mg/mL in 100% isopropyl alcohol. Formalin (10%) was pipetted onto the cells and mixed by gently rotating. Cells were incubated with formalin for 30 minutes. The formalin was then discarded, and the cells were washed twice with water, followed by the addition of 60% isopropanol, to which the cells were incubated for 5 minutes. After discarding the 60% isopropanol, Oil Red O Working Solution was evenly covered over the cells, which were allowed to incubate for 10-20 minutes. The Oil Red O solution was then discarded, and the cells were washed 3 times with water until no excess stain was seen. Hematoxylin was added to the cells and incubated for 1 minute, after which it was discarded, and the cells were washed 3 times with water. The cells were then covered with water and viewed under a 40× objective.

### Example 5—Wound Closure Over Time with WT or Mutated HOXA3

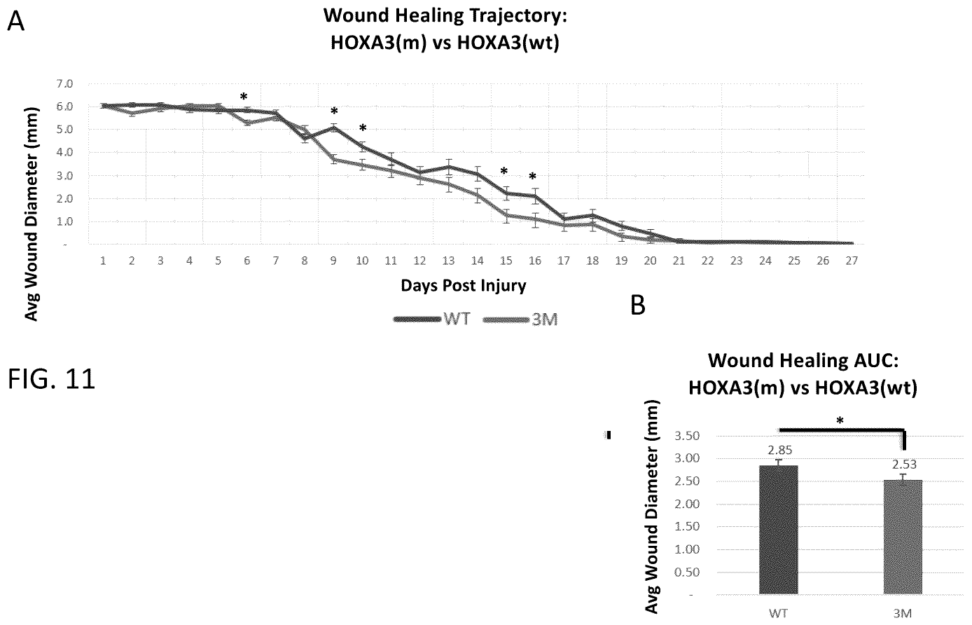

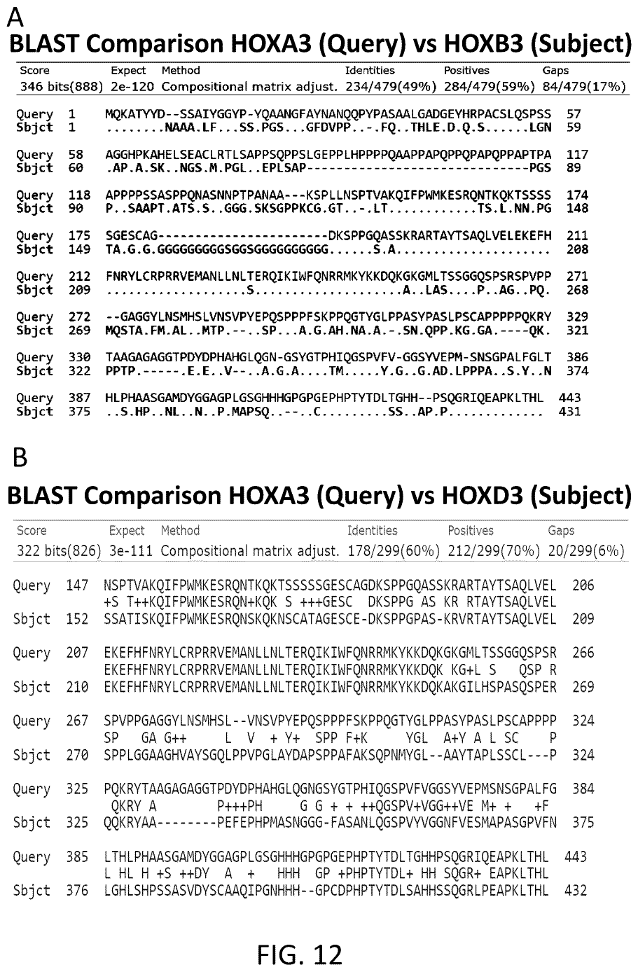

HOXA3 plasmid was modified to produce a modified HOXA3 protein with the following substitutions: L175D, E176K, and E178K. The resulting plasmid is referred to as HOXA3 (m) plasmid. The ability of HOXA3 (m) plasmid to promote wound closure in diabetic mice was compared to wild-type HOXA3 as described above. FIG. 11 shows results from these studies indicating an approximately 13% faster closure time as measured by average wound diameter at each time point, despite using the best known wildtype HoxA3 dosing protocol from applican'ts prior studies.

### Materials and Methods

### Methods

Diabetic Mice: Male B6.Cg-m+/+ Leprdb/J mice aged 8-12 weeks at start of study were obtained from Jackson Laboratories (Strain Code 000697). Mice were singly housed, and acclimated on-site for approximately one week prior to study activities. Mice were housed on a 12-hour light-dark cycle at 20-24 C (68-74 F) and 30-70% humidity. Mice were fed water and diet ad libitum throughout the study. Mice were randomized into 2 treatment groups based on blood glucose values prior to surgical wounding.

Aged Mice: Male and female C57BL/6J mice (77 weeks) were obtained from Jackson Laboratories. Mice were independently housed and acclimated on-site, for approximately one week prior to study activities. Mice were housed as described above. Mice were randomized into 2 treatment groups based on blood glucose values prior to surgical wounding.

Injury: All mice received two full thickness (through the panniculus) 6 mm wounds over the shoulders using a punch. These wounds were splinted with silicone splints, non-absorbable suture, and surgical glue to prevent skin contracture. Sample video of the wounding procedure is available in the Supplemental Data. Animals received once daily treatment with a non-steroidal anti-inflammatory for three days following wounding.

Treatment: Methylcellulose wafers were produced by mixing 25 μg (or lower where indicated) HoxA3 plasmid DNA in 1% methylcellulose and spotted in 50 μl droplets onto PARAFILM using a positive-displacement pipette. Sample video of wafer formation is provided in the Supplemental Data. Wafers were allowed to air dry a maximum of 5 hours prior to application, allowing them to reach a solid consistency. Plasmid wafers were prepared just prior to treatment. The sequence for the HoxA3 plasmid is provided in the supplemental information.

Wound Size Measurements: Wounds were measured using digital calipers under isoflurane anesthesia. Measurements of each wound were taken following the below (FIG. 1) four orientations and averaged for a single animal. Dual wounds on the same animal were generally uncorrelated and were thus treated as statistically independent.

Histology: Wounds and surrounding area were excised from the mice and fixed in 10% neutral buffered formalin for 48 hours. The wounds were then dehydrated by submerging in the following solutions: 70% ethanol (2 changes 1 hour each), 85% ethanol (2 changes 1 hours each), 95% ethanol (2 changes 1 hour each), 100% ethanol (2 changes 1 hour each), Histoclear xylene substitute for 3 hours, 3 hours of liquid paraffin (3 total changes 1 hour each). The tissues were placed in molds with the liquid paraffin wax, which was allowed to solidify on ice. Paraffin blocks were stored at room temperature until sectioning. Tissues were sectioned at a thickness of 5 μm to adhere onto microscope slides. Samples were placed on a 38° C. slide warmer until moisture disappeared. The paraffin wax was melted at 65° C. for 20 minutes prior to deparaffinization and subsequent staining.

Mason's Trichome: Slides were incubated with 55° C. Bouin's solution for 60 minutes immediately after slides were deparaffinized and rehydrated with distilled water. The slides were allowed to cool for 10 minutes after the 60-minute incubation with Bouin's solution. After equilibrating to room temperature, the tissues were rinsed with water until completely clear. Slides were then incubated with hematoxylin for 5 minutes, then immediately rinsed with tap water for 2 minutes. Slides were then incubated with a Biebrich Scarlet/Acid Fuchsin solution for 15 minutes, then immediately washed with tap water. Slides were then incubated with a Phosphomolybdic/Phosphotungstic Acid solution for 15 minutes. Without washing, slides were incubated with an analine blue solution for 10 minutes, then washed with deionized water. Slides were then incubated with 1% acetic acid solution for 5 minutes. After the incubation with acetic acid, slides were quickly cleared in 2 changes of 95% ethanol, followed by a quick exchange into xylene substitute. Slides were then mounted with toluene mounting media.

H+E Staining: Slides were deparaffinized with two 5-minute incubations of histoclear (xylene substitute) followed by two changes of 100% ethanol and two changes of 95% ethanol (3 minutes and 2 minutes respectively). Slides were then rehydrated with tap water for 1 minute. Slides were then stained with hematoxylin for 2 minutes followed by two separate 45 second rinses with tap water. Slides were then subjected to bluing reagent for 15 seconds. After bluing reagent, slides were washed twice with tap water for 30 seconds. Slides were then washed in 100% ethanol for 10 seconds, followed by a 3-minute incubation with Eosin Y for 3 minutes. After incubating with Eosin Y, slides were dehydrated with 4 changes of 100% ethanol. Slides were then cleared with two consecutive 3-minute incubations with histoclear. The slides were then mounted with toluene mounting media.

Histopathology Scoring: Anonymized samples were scored by a third-party board-certified pathologist. Criteria for pathological scoring of histology slides are as follows. Vascular Density, the number of vessels which contain visible blood cells and are lined by endothelium. Max Epithelial Thickness, number of cell layers of the epidermis in area of wound or open lesion. Fibroblast Cell Density, determined by cell density and surrounding extra-cellular matrix gauged with Mason' Trichrome. Granulation tissue formation, presence of newly formed blood vessels. Overall inflammation was also assessed. The numbers for vascular density and max epithelial thickness are total counts, while the other criteria are a relative severity score ranging from 1-5.

Statistics and Data: A third party statistician was sent anonymized raw data. Each figure is annotated with relevant statistical analysis. Annotated data and statistical analysis were performed with GraphPad Prism 9.

### Example 6—HOXA3 Protein Production

TAT-HOXA3 (m) and TAT-HOXA3 (wt) were produced, purified, and tested in culture. In some cases, production of HOXA3 in bacterial systems may be difficult, thus Applicants expressed the disclosed proteins in 293T cells.

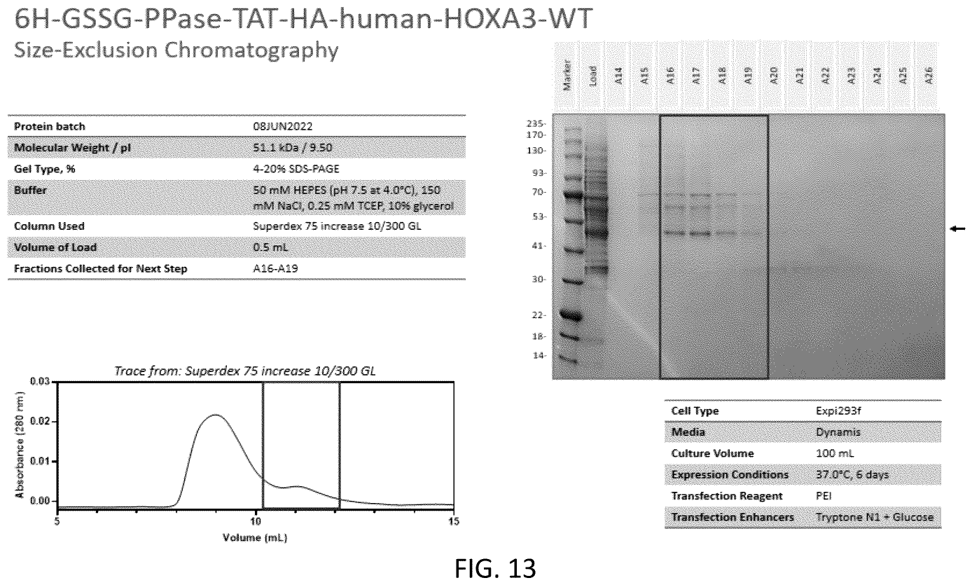

Briefly, transfected cells were collected, lysed, and clarified. Metal affinity, cation-exchange, and size-exclusion were sequentially employed to purify the HOXA3 proteins. The protein manufacturing procedure is provided in more detail in FIG. 13.

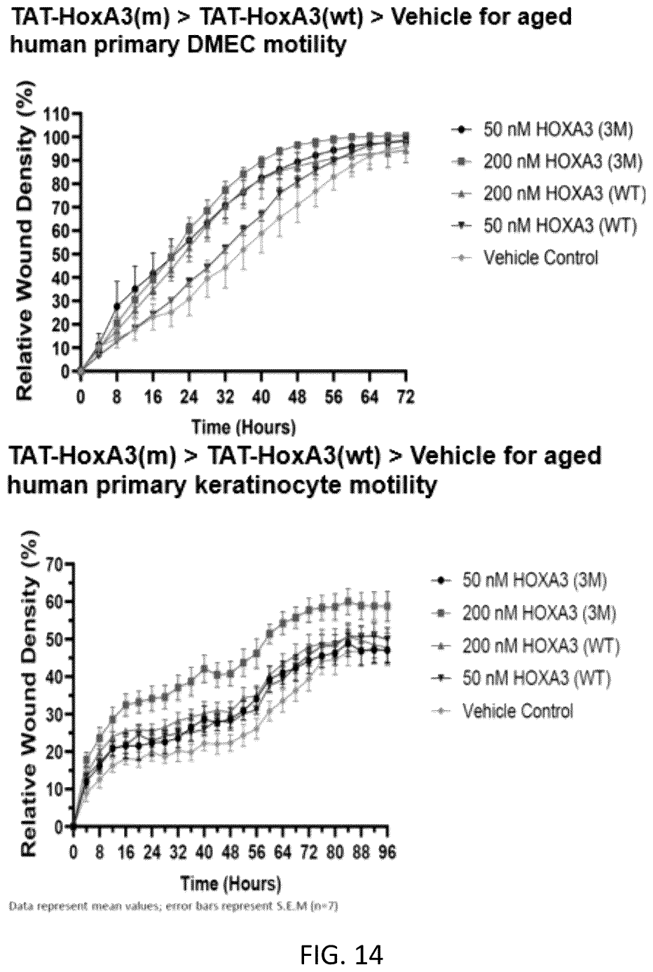

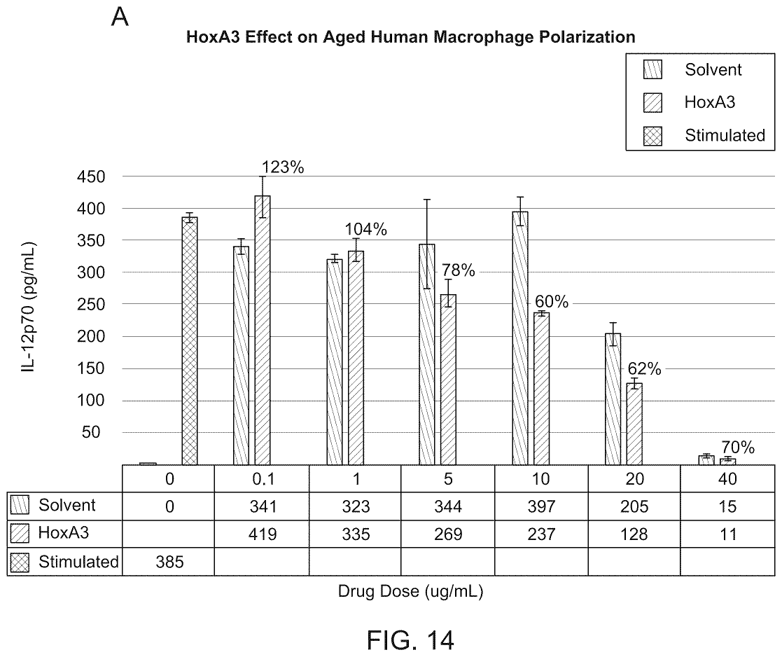

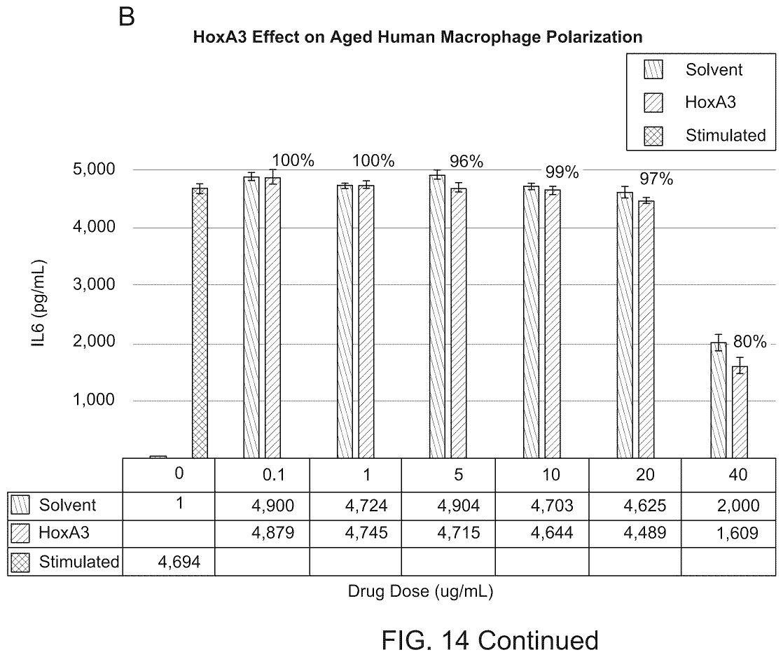

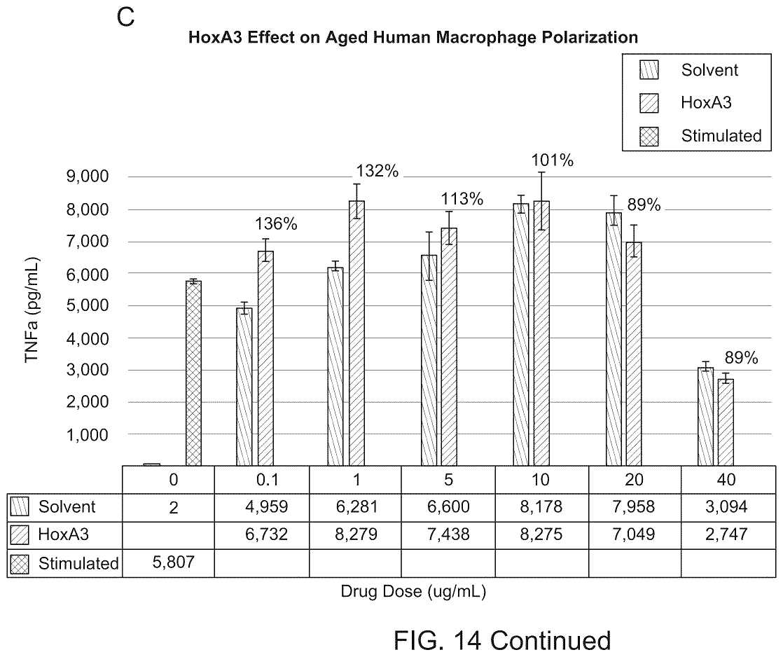

Protein purity was confirmed using chromatography, FIG. 14 (lower left corner) and gel electrophoresis (upper right corner). The fractions collected and pooled from size-exclusion chromatography were analyzed by gel electrophoresis and showed a strong band at the proper molecular weight of ~50 kD. It is important to note that the western blot analysis of the samples was after the proteins had been concentrated, frozen, and thawed. This means there is a significant amount of protein at the correct molecular weight, after freeze/thaw.

Bioactivity of the HOXA3 proteins produced in 293 cells was confirmed in relevant model cells—primary dermal human microvascular endothelial cells (hDMECs) from a 55 year old patient. The elderly hDMECs were found to grow substantially slower than younger hDMECs, which required several cycles of seeding optimization. Ultimately, 8,000 cells were seeded into 6-well plates, and allowed to adhere overnight prior to assay initiation. A scratch defect was created in the monolayer using a p200 pipette tip, then closure was monitored over time. Images of the scratched area were captured at regular intervals of 4 hours.

To quantify the rate of wound closure, the density of the “wound” was measured for cell confluency as a function of time. Analysis was complete using the IncuCyte S3 kinetic scratch wound analysis module.

Untreated cells migrated and filled the void more slowly than HOXA3-treated DMECs in a dose-dependent manner (FIG. 15).

### Example 7—HOXA3 Effect on Aged Human Macrophage Polarization

Human peripheral blood derived monocytes were cultured in ImmunoCult™-SF Macrophage Medium supplemented with M-CSF for 6 days to differentiate to M0 macrophages, then stimulated for approximately 48 hours with LPS/IFN-γ to generate M1 macrophages. The effect of TAT-HOXA3 (wt) and TAT-HOXA3 (m) on IL-6, IL-12p70, and TNFα cytokine production by LPS/IFN-γ activated M1 macrophages was assessed by pre-incubating M-CSF derived M0 macrophages with test articles for 30 minutes prior to the addition of stimulants for 2 days of activation. The secretion of IL-6, IL-12p70, TNFα in cell culture supernatants was characterized by MESOSCALE Discovery Panels. Bar graphs in FIG. 16 show results of TAT-HOXA3 (m), which are very similar to those obtained with TAT-HOXA3 (wt).

Cell Thawing, Plating, and M0 Differentiation (Day 0): On the day of culture set-up (Day 0), frozen human peripheral blood monocytes from one donor were thawed, washed, and assessed for viability using the Nexcelom Cellometer Auto 2000 Cell Profiler and AO/PI viability stain as outlined above. Cells were resuspended to 1×10⁶ cells/mL in ImmunoCult™-SF Macrophage Medium containing 50 ng/ml M-CSF (Shenandoah, Cat #100-03-10UG) and plated at 1×10⁵ cells/well (100 μL/well) in 96-well tissue culture treated plates (Costar, Cat #3595) to initiate the cultures. Triplicate cultures were set up for each condition.

Media Replenishment and Supplementation (Day 3): On day 3, fresh ImmunoCult™-SF Macrophage Medium containing 50 ng/ml M-CSF was prepared. Each well was topped up with 50 μL (50% volume) of the freshly prepared medium, gently mixed, returned to the incubator and cultured at 37° C., 5% CO₂ for an additional 3 days.

Macrophage Stimulation and Test Article Treatment (Day 6): Solvent control and test article working stock solutions (5× stocks) were prepared as described in Example 6 and added to appropriate Day 6 treatment cultures (40 μL/well). Solvent is 50 mM HEPES (pH 7.5 @ 4° C.), 500 mM NaCl, 5% glycerol. The cells were rested in the presence of test article or solvent for 30 minutes by incubation at 37° C., 5% CO₂. After the 30 minute rest, 10 μL/well of a 20× stock solution of LPS (Sigma, Cat #L6529) and IFN-γ (Shenandoah, Cat #100-77-20UG) was added to the appropriate wells for a final concentration of 10 ng/mL LPS and 50 ng/ml IFN-γ in a final volume of 200 μL/well and the cultures incubated at 37° C., 5% CO₂ for 2 days. For the unstimulated control cultures (see leftmost bars in Panels of FIG. 16), 50 μL/well of ImmunoCult™SF Macrophage Medium was added in place of LPS/IFN-γ and test article or solvent. All conditions were set up in triplicate.

MSD Supernatant Collection (Day 8) On Day 8, after 2 days of stimulation, the supernatants were transferred to new 96-well round bottom polypropylene plates (Costar, Cat #3879), centrifuged at 1500 rpm for 5 minutes to clear the supernatants, then the supernatants were carefully transferred as two ~90 μL aliquots into two new 96-well round bottom plates. The supernatant plates were sealed with Parafilm and stored at −80° C. until ready for MSD analysis.

One replicate of the 40 μg/mL and 10 μg/mL conditions for TAT-HOXA3 (wt) had severe bacterial contamination and were therefore not collected for downstream analysis. MESO SCALE DISCOVERY Analysis of Cytokine Expression in Culture Supernatants Custom V-Plex Proinflammatory Panel kits (MSD, Cat #K151A9H-1, K151QWD-1) were used for cytokine quantification of IL-6, IL-12p70, and TNFα. Calibrators, wash buffer, read buffer, and detection antibody solution were prepared according to the kit instructions. For IL-6, IL-12p70, and TNFα, on the day of the assay, the culture supernatant samples were brought to room temperature, and were run at a 1:2 dilution. Due to TNFα samples quantifying above the standard curve at the 1:2 dilution, a repeat of the TNFα assay was performed at a 1:50 dilution in PBS containing 1% BSA. The samples and calibrators were processed according to the kit instructions. Briefly, plates were prepared by washing three times with wash buffer (PBS+0.05% Tween-20 [SIGMA, Cat #P1379]), then supernatants and calibrators were added to the plates, and incubated at room temperature for 2 hours with shaking. Plates were then washed three times with wash buffer, then the detection antibodies were added and incubated at room temperature for 2 hours with shaking. Plates were then washed three times with wash buffer, 2× Read Buffer T was added, and the plates were read on a MESO QUICKPLEX SQ 120 Instrument. Analysis and Calculations The concentration of IL-6, IL-12p70, and TNFα was determined by interpolating the instrument response for each sample to the appropriate standard curve using the MSD Discovery Workbench 4.0 software, then applying the sample dilution factor. The effect of the solvent controls was determined by calculating the absolute cytokine expression of each solvent condition relative to the standard control (media only) and expressed as a fold change of standard control. The effect of each test article was determined by calculating the absolute cytokine expression of each test article condition relative to the closest matching solvent control and expressed as a fold change of solvent control. The solvent and test article conditions were graphed in GraphPad Prism version 9.1.

IL-12p70 showed a dose dependent suppression in response to HOXA3 (m) (shown) and HOXA3 (wt) (not shown) versus vehicle and buffer controls. At the higher concentrations of drug (essentially HOXA3 (m) or solvent, as solvent alone, at high concentration, was found to have an effect), IL12-p70 secretion was suppressed even for vehicle implying vehicle toxicity, but the suppression of IL-12p70 was still statistically significant for HOXA3-treated versus vehicle samples at these dosages. IL-6 expression was unaffected by TAT-HOXA3 (m) (shown) or TAT-HOXA (wt) (not shown). TNFα secretion was difficult to interpret as the ratio of HOXA3-treated TNFα secretion versus solvent decreases at higher dosages but the absolute secretion was unaffected. Secretion profiles are shown in FIG. 16 Panels A, B, and C. Note that for each panel the leftmost bar shows secretion from unstimulated cells, which are show basically no release of any chemokine with either solvent or HOXA3, and the black bar to the right of the unstimulated shows secretion with stimulation but no solvent or HOXA3.

### REFERENCES

[CDC 2020] National diabetes statistics report 2020: estimates of diabetes and its burden in the United States. www.cdc.gov/diabetes/pdfs/data/statistics/national-diabetes-statisticsreport.pdf (last accessed Sep. 16, 2022)
[Rice 2014] Rice J B, Desai U, Cummings A K G, Birbaum H G, Skornicki M, Parsons N. Burden of Venous Leg Ulcers in the United States. J Med Econ 2014; 17:5, 347-56.
[Baba 2015] Baba M, Davis W, Norman P, Davis T. Temporal Changes in the Prevalence and Associates of Foot Ulceration in Type 2 Diabetes: the Fremantle Diabetes Study. J Diabetes Complications 2015; 29:356-61.
[Tresierra-Ayala 2017] Tresierra-Ayala M A, Rojas A G. Association between Peripheral Arterial Disease and Diabetic Foot Ulcers in Patients with Diabetes Mellitus Type 2. Medicina Universitaria 2017; 19 (76): 123-6.
[Prompers 2007] Prompers L, Huijberts M, Apelqvist J, Jude E, Piaggesi A, Bakker K, Edmonds M, Holstein P, Jirkovska A, Mauricio D, Tennvall G R, Reike H, Spraul M, Uccioli L, Urbancic V, Van Acker K, Baal Jv, Merode Fv, Schaper N. High Prevalence of Ischaemia, Infection and Serious Comorbidity in Patients with Diabetic Foot Disease in Europe. Baseline results from the Eurodiale Study. Diabetologia 2007; 50:18-25.
[Lipsky 2012] Lipsky B A, Berendt A R, Cornia P B, Pile J C, Peters E J G, Armstrong D G, Deery H G, Embil J M, Joseph W S, Karchmer A W, Pinzur M S, Senneville E. 2012 Infectious Diseases Society of America Clinical Practice Guideline for the Diagnosis and Treatment of Diabetic Foot Infections. Clin Infect Dis 2012; 54: e132-73.
[Kruse 2006] Kruse I, Edelman S. Evaluation and Treatment of Diabetic Foot Ulcers. Clinical Diabetes 2006; 24 (2): 91-3.
[Olsson 2019] Olsson M, Jarbrink K, Divakar U, Bajpai R, Upton Z, Schmidtchen A, Car J. The Humanistic and Economic Burden of Chronic Wounds: A Systematic Review. Wound Rep Reg 2019; 27:114-25.
[Jeffcoate 2018] Jeffcoate W J, Vileikyte L, Boyko E J, Armstrong D G, Boulton A J M. Current Challenges and Opportunities in the Prevention and Management of Diabetic Foot Ulcers. Diabetes Care 2018; 41:645.
[ORGO 2018] Organogenesis. (2019 Dec., 31). Form 10-K. Retrieved from investors.organogenesis.com
[Tettelbach 2019] Tettelbach W, Cazzell S, Sigal F, Caporusso J M, Agnew P S, Hanft J, Dove C. A Multicentre Prospective Randomized Controlled Comparative Parallel Study of Dehydrated Human Umbilical Cord (Epicord) Allograft for the Treatment of Diabetic Foot Ulcers. Int Wound J 2019; 16:122-30.
[Haugh 2017] Haugh A M, Witt J G, Hauch A, Darden M, Parker G, Ellsworth W A, Buell J F. Amnion Membrane in Diabetic Foot Wounds: a Meta-analysis. Plast Reconst Surg Glob Open 2017; 5: e1302.
[Armstrong 2017] Armstrong D G, Boulton A J M, Bus S A. Diabetic Foot Ulcers and Their Recurrence. N Engl J Med 2017; 376:2367-75.
[Zelen 2016] Zelen C M, Serena T E, Gould L, Le L, Carter M J, Keller J, Li W W. Treatment of Chronic Diabetic Lower Extremity Ulcers with Advanced Therapies: a Prospective, Randomised, Controlled, Multi-centre Comparative Study Examining Clinical Efficacy and Cost. Int Wound J. 2016; 2:272-82.
[Blume 2014] Blume P, Bowlby M, Schmidt B M, Donegan R. Safety and Efficacy of Becaplermin Gel in the Treatment of Diabetic Foot Ulcers. Chronic Wound Care Management Res 2014; 1:11-4.
[Lopez-Saura 2013] Lopez-Saura P, Year-Alos I B, Valenzuela-Silva C, Gonzalez-Diaz O, del Rio-Martin A, Berlanga-Acosta J, Fernandez-Montequin J I, Acevedo-Castro B, Lopez-Mola E, Herrera-Martinez L. Medical Practice Confirms Clinical Trial Results of the Use of Intralesional Human Recombinant Epidermal Growth Factor in Advanced Diabetic Foot Ulcers. Adv Pharmacoepidem Drug Safety 2013; 2 (2): 1-9.
[Grek 2015] Grek C L, Prasad G M, Viswanathan V, Armstrong D G, Gourdie R G, Ghatnekar G S. Topical Administration of a Connexin43-based Peptide Augments Healing of Chronic Neuropathic Diabetic Foot Ulcers: a Multicenter, Randomized Trial, Wound Rep Reg 2015; 23:202-12.
[Barrientos 2008] Barrientos S, Stojadinovic O, Golinko M S, Brem H, Tomic-Canic M. Growth Factors and Cytokines in Wound Healing. Wound Rep Reg 2008; 16:585-601.
[Freedberg 2001] Freedberg I M, Tomic-Canic M, Komine M, Blumenberg M. Keratins and the Keratinocyte Activation Cycle 2001; 116 (5): 633-40.
[Brem 2007] Brem H, Tomic-Canic M. Cellular and Molecular Basis of Wound Healing in Diabetes. J Clin Invest 2007; 117:1219-22.
[Okonkwo 2017] Okonkwo U A, DiPietro L A. Diabetes and Wound Angiogenesis. Int J Mol Sci 2017; 18 (1419): 1-15.
[Mast 1996] Mast B A, Schultz G S. Interactions of Cytokines, Growth Factors, and Proteases in Acute and Chronic Wounds. Wound Repair Regen 1996; 4:411-20.
[Veith 2019] Veith A P, Henderson K, Spencer A, Sligar A D, Baker A B. Therapeutic Strategies for Enhancing Angiogenesis in Wound Healing. Adv Drug Deliv Rev 2019; 146:97-125.
[Serra 2017] Serra M B, Barroso W A, da Silva N N, Silva SdN, Borges A C R, Abreu I C, Borges M OdR. From Inflammation to Current and Alternative Therapies Involved in Wound Healing. Int J Inflam 2017, 1-17.
[Kim 2019] Kim S Y, Nair M G. Macrophages in Wound Healing: Activation and Plasticity. Immunol Cell Biol 2019; 97:258-67.
[Mace 2005] Mace K A, Hansen S L, Myers C, Young D M, Boudreau N J. HOXA3 Induces Cell Migration in Endothelial and Epithelial Cells Promoting Angiogenesis and Wound Repair. J Cell Sci 2005; 118:2567-77.
[Mace 2009] Mace K A, Restivo T E, Rinn J L, Paquet A C, Chang H Y, Young D M, Boudreau N J. HOXA3 Modulates Injury-induced Mobilization and Recruitment of Bone Marrow-derived Cells. Stem Cells 2009; 27:1654-65.
[Sadoun 2016] Sadoun H A, Burgess M, Hentges K E, Mace K A. Enforced Expression of Hoxa3 Inhibits Classical and Promotes Alternative Activation of Macrophages In Vitro and In Vivo. J Immunol 2016; 197 (3): 8724.
[Mahdipour 2011] Mahdipour E, Charnock J C, Mace, K. A. Hoxa3 Promotes the Differentiation of Hematopoietic Progenitor Cells into Proangiogenic Gr-1+CD11b+ Myeloid Cells. Blood 2011; 117:815-26.
[Alrdahe 2019] Alrdahe S, Sadoun H A, Torbica T, McKenzi E A, Bowling F L, Boulton A J, Mace K A. Dysregulation of Macrophage Development and Phenotype in Diabetic Human Macrophages can be Rescued by HOXA3 Protein Transduction. PLOS ONE 2019; 14 (10): e0223980.
[Kuo 2014] Kuo J H, Cuevas I, Chen A, Dunn A, Kuri M, Boudreau N. Secreted HoxA3 Promotes Epidermal Proliferation and Angiogenesis in Genetically Modified Three-Dimensional Composite Skin Constructs. Adv Wound Care (New Rochelle) 2013; 3:605-13.
[Kachgal 2012] Kachgal S, Mace K A, Boudreau N J. The Dual Roles of Homeobox Genes in Vascularization and Wound Healing. Cell Adh Migr 2012; 6:457-70.
[Gould 2015] Gould L, Abadir P, Brem H, Carter M, Conner-Kerr T, Davidson J, DiPietro L, Falanga V, Fife C, Gardner S, Grice E, Harmon J, Hazzard W R, High K P, Houghton P, Jacobson N, Kirsner, R S, Kovacs E J, Margolis D, Horne F M, Reed R J, Sullivan D H, Thom S, Tomic-Canic M, Walston J, Whitney J A, Williams J, Zieman S, Schmader K. Chronic Wound Repair and Healing in Older Adults: Current Status and Future Research. JAGS 2015; 63:427-38.
[Moura 2019] Moura J, Maureira P, Leal E C, Fonseca A C, Carvalho E. Immune Aging in Diabetes and its Implications in Wound Healing. Clin Immunol 2019; 200:43-54.
[Hansen 2003] Hansen S L, Myers C A, Charboneau A, Young D M, Boudreau N. HoxD3 Accelerates Wound Healing in Diabetic Mice. Am J Pathol 2003; 163 (6): 2421-32.
[Makrantonaki 2017] Makrantonaki E, Wlaschek M, Scharffetter-Kochanek K. Pathogenesis of Wound Healing Disorders in the Elderly. Deutsche Dermatol Gesellschaft 2017; 255-75.
[Blakytny 2006] Blakytny R, Jude E. The Molecular Biology of Chronic Wounds and Delayed Healing in Diabetes. Diabetic Med 2006; 23:594-608.
[Sen 2021] Sen C K. Human Wound and Its Burden: Updated 2020 Compendium of Estimates. Adv Wound Care 2021; 10 (5): 281-292.
[Nussbaum 2017] Nussbaum S R, Carter M J, Fife C E, DaVanzo J, Haught R, Nusgart M, Cartwright D. An Economic Evaluation of the Impact, Cost, and Medicare Policy Implications of Chronic Nonhealing Wounds. Value Health 2017; 1-6.
[Graves 2014] Graves N, Zheng H. The Prevalence and Incidence of Chronic Wounds: a Literature Review. Wound Practice Res 2014; 22 (1): 1-19.
[Darwin 2018] Darwin E, Tomic-Canic M. Healing Chronic Wounds: Current Challenges and Potential Solutions. Current Dermatol Rep 2018; 1-7.
[Malone 2017] Malone M, Bjarnsholt T, McBain A J, James G A, Stoodley P, Leaper D, Tachi M, Schultz G, Swanson T, Wolcott R D. The Prevalance of Biofilms in Chronic Wounds: a Systematic Review and Meta-analysis of Published Data. J Wound Care 2017; 26 (1): 20-5.
[Franceschi 2018] Franceschi C, Garagnani P, Parini P, Giuliani C, Santoro A. Inflammaging: a New Immune-Metabolic Viewpoint for Age-Related Diseases. Nature Reviews Endocrinol 2018; 14:57690.
[Buikema 2014] Buikema K E S, Meyerle J H. Amputation Stump: Privileged Harbor for Infections, Tumors, and Immune Disorders. Clinics in Dermatol 2014; 32:670-7.
[Liu 2019] Liu M A. A Comparison of Plasmid DNA and mRNA as Vaccine Technologies. Vaccines 2019; 7 (37): 1-20.
[Ma 2020] Ma C C, Wang Z L, Xu T, He Z Y, Wei Y Q. The Approved Gene Therapy Drugs Worldwide: from 1998-2019. Biotechnol Adv 2020; 1-14.
[Morgan 2021] Morgan R, Hunter K, Pandha H S. Downstream of the HOX Genes: Explaining Conflicting Tumour Suppressor and Oncogenic Functions in Cancer. Int J Cancer 2022; 1-14.
[Zhou 2013] Lee J, Shieh J H, Zhang J, Liren L, Zhang Y, Eom J Y, Morrone G, Moore M A S, Zhou P. Improved ex vivo Explansion of Adult Hematopoietic Stem Cells by Overcoming CUL4-mediated Degradation of HOXB4. Blood 2013; 121 (20): 4082-9.
[Truzzi 2020] Truzzi E, Nascimento T L, lannuccelli V, Constantino L, Lima E M, Leo E, Siligardi C, Gualtieri M L, Maretti E. In vivo Biodistribution of Respirable Solid Lipid Nanoparticles Surface-Decorated with a Mannose-Based Surfactant: A Promising Tool for Pulmonary Tuberculosis Treatment. Nanomaterials 2020; 10:568-83.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed description is to be regarded as illustrative in nature and not restrictive.

All references disclosed herein, whether patent or non-patent, are hereby incorporated by reference as if each was included at its citation, in its entirety. In case of conflict between reference and specification, the present specification, including definitions, will control.

Although the present disclosure has been described with a certain degree of particularity, it is understood the disclosure has been made by way of example, and changes in detail or structure may be made without departing from the spirit of the disclosure as defined in the appended claims.

HOXA3-available at uniprot.org/uniprot/043365
SEQ ID NO: 1
MQKATYYDSSAIYGGYPYQAANGFAYNANQQPYPASAALGADGEYHRPACSLQSPSSAGGHPKAHELSEACLRT
LSAPPSQPPSLGEPPLHPPPPQAAPPAPQPPQPAPQPPAPTPAAPPPPSSASPPQNASNNPTPANAAKSPLLNS
PTVAKQIFPWMKESRQNTKQKTSSSSSGESCAGDKSPPGQASSKRARTAYTSAQLVELEKEFHFNRYLCRPRRV
EMANLLNLTERQIKIWFQNRRMKYKKDQKGKGMLTSSGGQSPSRSPVPPGAGGYLNSMHSLVNSVPYEPQSPPP
FSKPPQGTYGLPPASYPASLPSCAPPPPPQKRYTAAGAGAGGTPDYDPHAHGLQGNGSYGTPHIQGSPVFVGGS
YVEPMSNSGPALFGLTHLPHAASGAMDYGGAGPLGSGHHHGPGPGEPHPTYTDLTGHHPSQGRIQEAPKLTHL
HOXB3-available at uniprot.org/uniprot/P14651
SEQ ID NO: 2
MQKATYYDNAAAALFGGYSSYPGSNGFGFDVPPQPPFQAATHLEGDYQRSACSLQSLGNAAPHAKSKELNGSCM
RPGLAPEPLSAPPGSPPPSAAPTSATSNSSNGGGPSKSGPPKCGPGTNSTLTKQIFPWMKESRQTSKLKNNSPG
TAEGCGGGGGGGGGGGSGGSGGGGGGGGGGDKSPPGSAASKRARTAYTSAQLVELEKEFHFNRYLCRPRRVEMA
NLLNLSERQIKIWFQNRRMKYKKDQKAKGLASSSGGPSPAGSPPQPMQSTAGFMNALHSMTPSYESPSPPAFGK
AHQNAYALPSNYQPPLKGCGAPQKYPPTPAPEYEPHVLQANGGAYGTPTMQGSPVYVGGGGYADPLPPPAGPSL
YGLNHLSHHPSGNLDYNGAPPMAPSQHHGPCEPHPTYTDLSSHHAPPPQGRIQEAPKLTHL
HOXD3-uniprot.org/uniprot/P31249
SEQ ID NO: 3
MLFEQGQQALELPECTMQKAAYYENPGLFGGYGYSKTTDTYGYSTPHQPYPPPAAASSLDTDYPGSACSIQSSA
PLRAPAHKGAELNGSCMRPGTGNSQGGGGGSQPPGLNSEQQPPQPPPPPPTLPPSSPTNPGGGVPAKKPKGGPN
ASSSSATISKQIFPWMKESRQNSKQKNSCATAGESCEDKSPPGPASKRVRTAYTSAQLVELEKEFHFNRYLCRP
RRVEMANLLNLTERQIKIWFQNRRMKYKKDQKAKGILHSPASQSPERSPPLGGAAGHVAYSGQLPPVPGLAYDA
PSPPAFAKSQPNMYGLAAYTAPLSSCLPQQKRYAAPEFEPHPMASNGGGFASANLQGSPVYVGGNFVESMAPAS
GPVFNLGHLSHPSSASVDYSCAAQIPGNHHHGPCDPHPTYTDLSAHHSSQGRLPEAPKLTHL
Un-Expressed Leader Peptide Sequence
SEQ ID NO: 4
NYDSLGDPSWLVKLGTELGS
Terminal Peptide Sequence Myc with His tag
SEQ ID NO: 5
GPFEQKLISEEDLNMHTGHHHHHH
Genetic Nucleotide Sequence of Expression Plasmid
SEQ ID NO: 10
GACGGATCGGGAGATCTCCCGATCCCCTATGGTCGACTCTCAGTACAATCTGCTCTGATGCCGCATAGTTAAGC
CAGTATCTGCTCCCTGCTTGTGTGTTGGAGGTCGCTGAGTAGTGCGCGAGCAAAATTTAAGCTACAACAAGGCA
AGGCTTGACCGACAATTGCATGAAGAATCTGCTTAGGGTTAGGCGTTTTGCGCTGCTTCGCGATGTACGGGCCA
GATATACGCGTTGACATTGATTATTGACTAGTTATTAATAGTAATCAATTACGGGGTCATTAGTTCATAGCCCA
TATATGGAGTTCCGCGTTACATAACTTACGGTAAATGGCCCGCCTGGCTGACCGCCCAACGACCCCCGCCCATT
GACGTCAATAATGACGTATGTTCCCATAGTAACGCCAATAGGGACTTTCCATTGACGTCAATGGGTGGACTATT
TACGGTAAACTGCCCACTTGGCAGTACATCAAGTGTATCATATGCCAAGTACGCCCCCTATTGACGTCAATGAC
GGTAAATGGCCCGCCTGGCATTATGCCCAGTACATGACCTTATGGGACTTTCCTACTTGGCAGTACATCTACGT
ATTAGTCATCGCTATTACCATGGTGATGCGGTTTTGGCAGTACATCAATGGGCGTGGATAGCGGTTTGACTCAC
GGGGATTTCCAAGTCTCCACCCCATTGACGTCAATGGGAGTTTGTTTTGGCACCAAAATCAACGGGACTTTCCA
AAATGTCGTAACAACTCCGCCCCATTGACGCAAATGGGCGGTAGGCGTGTACGGTGGGAGGTCTATATAAGCAG
AGCTCTCTGGCTAACTAGAGAACCCACTGCTTACTGGCTTATCGAAATTAATACGACTCACTATAGGGAGACCC
AAGCTGGCTAGTTAAGCTTGGTACCGAGCTCGGATCCATGCAAAAAGCGACCTACTATGACAGCAGCGCGATCT
ACGGTGGCTACCCGTATCAGGCGGCGAACGGCTTCGCGTACAACGCGAACCAGCAACCGTACCCGGCGAGCGCG
GCGCTGGGTGCGGATGGTGAATATCACCGTCCGGCGTGCAGCCTGCAGAGCCCGAGCAGCGCGGGTGGCCACCC
GAAAGCGCATGAACTGAGCGAGGCGTGCCTGCGTACCCTGAGCGCTCCGCCGAGCCAGCCGCCGAGCCTGGGCG
AGCCGCCGCTGCACCCGCCGCCGCCGCAGGCGGCTCCGCCGGCGCCGCAGCCGCCGCAACCGGCGCCGCAACCG
CCGGCGCCGACCCCGGCGGCTCCGCCGCCGCCGAGCAGCGCGAGCCCGCCGCAGAACGCGAGCAACAACCCGAC
CCCGGCGAACGCGGCGAAAAGCCCGCTGCTGAACAGCCCGACCGTGGCGAAGCAAATTTTTCCGTGGATGAAAG
AAAGCCGTCAGAACACCAAGCAAAAAACCAGCAGCAGCAGCAGCGGTGAAAGCTGCGCGGGTGATAAAAGCCCG
CCGGGTCAGGCGAGCAGCAAACGTGCGCGTACCGCGTATACCAGCGCGCAACTGGACAAGCTGAAGAAAGAGTT
CCACTTTAACCGTTACCTGTGCCGTCCGCGTCGTGTTGAAATGGCGAACCTGCTGAACCTGACCGAGCGTCAGA
TCAAGATTTGGTTCCAAAACCGTCGTATGAAATACAAGAAAGATCAGAAGGGTAAAGGCATGCTGACCAGCAGC
GGTGGCCAAAGCCCGAGCCGTAGCCCGGTGCCGCCGGGTGCGGGTGGCTACCTGAACAGCATGCACAGCCTGGT
GAACAGCGTTCCGTATGAACCGCAAAGCCCGCCGCCGTTTAGCAAGCCGCCGCAAGGTACCTATGGTCTGCCGC
CGGCGAGCTATCCGGCGAGCCTGCCGAGCTGCGCGCCGCCGCCGCCGCCGCAGAAGCGTTACACCGCGGCGGGT
GCGGGCGCGGGTGGCACCCCGGACTATGATCCGCACGCGCATGGTCTGCAGGGTAACGGTAGCTACGGCACCCC
GCACATCCAAGGTAGCCCGGTGTTCGTTGGTGGCAGCTACGTTGAACCGATGAGCAACAGCGGTCCGGCGCTGT
TTGGTCTGACCCACCTGCCGCATGCGGCGAGCGGTGCGATGGACTATGGTGGCGCGGGTCCGCTGGGTAGCGGT
CACCACCATGGTCCGGGTCCGGGTGAACCGCACCCGACCTACACCGATCTGACCGGTCACCACCCGAGCCAGGG
TCGTATTCAAGGGCCCTTCGAACAAAAACTCATCTCAGAAGAGGATCTGAATATGCATACCGGTCATCATCACC
ATCACCATTGAGTTTAAACCCGCTGATCAGCCTCGACTGTGCCTTCTAGTTGCCAGCCATCTGTTGTTTGCCCC
TCCCCCGTGCCTTCCTTGACCCTGGAAGGTGCCACTCCCACTGTCCTTTCCTAATAAAATGAGGAAATTGCATC
GCATTGTCTGAGTAGGTGTCATTCTATTCTGGGGGGTGGGGTGGGGCAGGACAGCAAGGGGGAGGATTGGGAAG
ACAATAGCAGGCATGCTGGGGATGCGGTGGGCTCTATGGCTTCTGAGGCGGAAAGAACCAGCTGGGGCTCTAGG
GGGTATCCCCACGCGCCCTGTAGCGGCGCATTAAGCGCGGCGGGTGTGGTGGTTACGCGCAGCGTGACCGCTAC
ACTTGCCAGCGCCCTAGCGCCCGCTCCTTTCGCTTTCTTCCCTTCCTTTCTCGCCACGTTCGCCGGCTTTCCCC
GTCAAGCTCTAAATCGGGGCATCCCTTTAGGGTTCCGATTTAGTGCTTTACGGCACCTCGACCCCAAAAAACTT
GATTAGGGTGATGGTTCACGTAGTGGGCCATCGCCCTGATAGACGGTTTTTCGCCCTTTGACGTTGGAGTCCAC
GTTCTTTAATAGTGGACTCTTGTTCCAAACTGGAACAACACTCAACCCTATCTCGGTCTATTCTTTTGATTTAT
AAGGGATTTTGGGGATTTCGGCCTATTGGTTAAAAAATGAGCTGATTTAACAAAAATTTAACGCGAATTAATTC
TGTGGAATGTGTGTCAGTTAGGGTGTGGAAAGTCCCCAGGCTCCCCAGGCAGGCAGAAGTATGCAAAGCATGCA
TCTCAATTAGTCAGCAACCAGGTGTGGAAAGTCCCCAGGCTCCCCAGCAGGCAGAAGTATGCAAAGCATGCATC
TCAATTAGTCAGCAACCATAGTCCCGCCCCTAACTCCGCCCATCCCGCCCCTAACTCCGCCCAGTTCCGCCCAT
TCTCCGCCCCATGGCTGACTAATTTTTTTTATTTATGCAGAGGCCGAGGCCGCCTCTGCCTCTGAGCTATTCCA
GAAGTAGTGAGGAGGCTTTTTTGGAGGCCTAGGCTTTTGCAAAAAGCTCCCGGGAGCTTGTATATCCATTTTCG
GATCTGATCAAGAGACAGGATGAGGATCGTTTCGCATGATTGAACAAGATGGATTGCACGCAGGTTCTCCGGCC
GCTTGGGTGGAGAGGCTATTCGGCTATGACTGGGCACAACAGACAATCGGCTGCTCTGATGCCGCCGTGTTCCG
GCTGTCAGCGCAGGGGCGCCCGGTTCTTTTTGTCAAGACCGACCTGTCCGGTGCCCTGAATGAACTGCAGGACG
AGGCAGCGCGGCTATCGTGGCTGGCCACGACGGGCGTTCCTTGCGCAGCTGTGCTCGACGTTGTCACTGAAGCG
GGAAGGGACTGGCTGCTATTGGGCGAAGTGCCGGGGCAGGATCTCCTGTCATCTCACCTTGCTCCTGCCGAGAA
AGTATCCATCATGGCTGATGCAATGCGGCGGCTGCATACGCTTGATCCGGCTACCTGCCCATTCGACCACCAAG
CGAAACATCGCATCGAGCGAGCACGTACTCGGATGGAAGCCGGTCTTGTCGATCAGGATGATCTGGACGAAGAG
CATCAGGGGCTCGCGCCAGCCGAACTGTTCGCCAGGCTCAAGGCGCGCATGCCCGACGGCGAGGATCTCGTCGT
GACCCATGGCGATGCCTGCTTGCCGAATATCATGGTGGAAAATGGCCGCTTTTCTGGATTCATCGACTGTGGCC
GGCTGGGTGTGGCGGACCGCTATCAGGACATAGCGTTGGCTACCCGTGATATTGCTGAAGAGCTTGGCGGCGAA
TGGGCTGACCGCTTCCTCGTGCTTTACGGTATCGCCGCTCCCGATTCGCAGCGCATCGCCTTCTATCGCCTTCT
TGACGAGTTCTTCTGAGCGGGACTCTGGGGTTCGCGAAATGACCGACCAAGCGACGCCCAACCTGCCATCACGA
GATTTCGATTCCACCGCCGCCTTCTATGAAAGGTTGGGCTTCGGAATCGTTTTCCGGGACGCCGGCTGGATGAT
CCTCCAGCGCGGGGATCTCATGCTGGAGTTCTTCGCCCACCCCAACTTGTTTATTGCAGCTTATAATGGTTACA
AATAAAGCAATAGCATCACAAATTTCACAAATAAAGCATTTTTTTCACTGCATTCTAGTTGTGGTTTGTCCAAA
CTCATCAATGTATCTTATCATGTCTGTATACCGTCGACCTCTAGCTAGAGCTTGGCGTAATCATGGTCATAGCT
GTTTCCTGTGTGAAATTGTTATCCGCTCACAATTCCACACAACATACGAGCCGGAAGCATAAAGTGTAAAGCCT
GGGGTGCCTAATGAGTGAGCTAACTCACATTAATTGCGTTGCGCTCACTGCCCGCTTTCCAGTCGGGAAACCTG
TCGTGCCAGCTGCATTAATGAATCGGCCAACGCGCGGGGAGAGGCGGTTTGCGTATTGGGCGCTCTTCCGCTTC
CTCGCTCACTGACTCGCTGCGCTCGGTCGTTCGGCTGCGGCGAGCGGTATCAGCTCACTCAAAGGCGGTAATAC
GGTTATCCACAGAATCAGGGGATAACGCAGGAAAGAACATGTGAGCAAAAGGCCAGCAAAAGGCCAGGAACCGT
AAAAAGGCCGCGTTGCTGGCGTTTTTCCATAGGCTCCGCCCCCCTGACGAGCATCACAAAAATCGACGCTCAAG
TCAGAGGTGGCGAAACCCGACAGGACTATAAAGATACCAGGCGTTTCCCCCTGGAAGCTCCCTCGTGCGCTCTC
CTGTTCCGACCCTGCCGCTTACCGGATACCTGTCCGCCTTTCTCCCTTCGGGAAGCGTGGCGCTTTCTCAATGC
TCACGCTGTAGGTATCTCAGTTCGGTGTAGGTCGTTCGCTCCAAGCTGGGCTGTGTGCACGAACCCCCCGTTCA
GCCCGACCGCTGCGCCTTATCCGGTAACTATCGTCTTGAGTCCAACCCGGTAAGACACGACTTATCGCCACTGG
CAGCAGCCACTGGTAACAGGATTAGCAGAGCGAGGTATGTAGGCGGTGCTACAGAGTTCTTGAAGTGGTGGCCT
AACTACGGCTACACTAGAAGGACAGTATTTGGTATCTGCGCTCTGCTGAAGCCAGTTACCTTCGGAAAAAGAGT
TGGTAGCTCTTGATCCGGCAAACAAACCACCGCTGGTAGCGGTGGTTTTTTTGTTTGCAAGCAGCAGATTACGC
GCAGAAAAAAAGGATCTCAAGAAGATCCTTTGATCTTTTCTACGGGGTCTGACGCTCAGTGGAACGAAAACTCA
CGTTAAGGGATTTTGGTCATGAGATTATCAAAAAGGATCTTCACCTAGATCCTTTTAAATTAAAAATGAAGTTT
TAAATCAATCTAAAGTATATATGAGTAAACTTGGTCTGACAGTTACCAATGCTTAATCAGTGAGGCACCTATCT
CAGCGATCTGTCTATTTCGTTCATCCATAGTTGCCTGACTCCCCGTCGTGTAGATAACTACGATACGGGAGGGC
TTACCATCTGGCCCCAGTGCTGCAATGATACCGCGAGACCCACGCTCACCGGCTCCAGATTTATCAGCAATAAA
CCAGCCAGCCGGAAGGGCCGAGCGCAGAAGTGGTCCTGCAACTTTATCCGCCTCCATCCAGTCTATTAATTGTT
GCCGGGAAGCTAGAGTAAGTAGTTCGCCAGTTAATAGTTTGCGCAACGTTGTTGCCATTGCTACAGGCATCGTG
GTGTCACGCTCGTCGTTTGGTATGGCTTCATTCAGCTCCGGTTCCCAACGATCAAGGCGAGTTACATGATCCCC
CATGTTGTGCAAAAAAGCGGTTAGCTCCTTCGGTCCTCCGATCGTTGTCAGAAGTAAGTTGGCCGCAGTGTTAT
CACTCATGGTTATGGCAGCACTGCATAATTCTCTTACTGTCATGCCATCCGTAAGATGCTTTTCTGTGACTGGT
GAGTACTCAACCAAGTCATTCTGAGAATAGTGTATGCGGCGACCGAGTTGCTCTTGCCCGGCGTCAATACGGGA
TAATACCGCGCCACATAGCAGAACTTTAAAAGTGCTCATCATTGGAAAACGTTCTTCGGGGCGAAAACTCTCAA
GGATCTTACCGCTGTTGAGATCCAGTTCGATGTAACCCACTCGTGCACCCAACTGATCTTCAGCATCTTTTACT
TTCACCAGCGTTTCTGGGTGAGCAAAAACAGGAAGGCAAAATGCCGCAAAAAAGGGAATAAGGGCGACACGGAA
ATGTTGAATACTCATACTCTTCCTTTTTCAATATTATTGAAGCATTTATCAGGGTTATTGTCTCATGAGCGGAT
ACATATTTGAATGTATTTAGAAAAATAAACAAATAGGGGTTCCGCGCACATTTCCCCGAAAAGTGCCACCTGAC
GTC

We claim:

1. A method of enhancing wound repair in a patient comprising:
identifying a wound in a patient;
applying a composition comprising HOXA3 to the wound; and
allowing the wound to heal.

2. The method of claim 1, wherein the HOXA3 is a selected from a nucleic acid sequence and an amino acid sequence.

3. The method of claim 1 or claim 2, wherein the HOXA3 is an mRNA coding for HOXA3.

4. The method of any one of claims 1-3, wherein the HOXA3 in comprised on a plasmid.

5. The method of any one of claims 1-4, wherein the HOXA3 includes a moiety for penetrating a cell.

6. The method of any one of claims 1-5, wherein the HOXA3 is at least about 49% identical to a wild-type HOXA3.

7. The method of any one of claim 1-6, wherein the composition includes a lipid nanoparticle comprising the HOXA3.

8. The method of any one of claims 1-7, wherein the patient is human.

9. The method of any one of claims 1-8, wherein the patient is selected from one or more of at least about 60 years of age, diabetic, suffering from immunoaging.

10. The method of any one of claims 1-9, wherein the patient is an amputee.

11. A system for enhancing wound repair in a patient, comprising:
an applicator; and
a composition comprising HOXA3.

12. The system of claim 11, wherein the HOXA3 is a coding sequence.

13. The system of claim 11 or claim 12, wherein the HOXA3 is an mRNA coding for HOXA3.

14. The system of any one of claims 11-12, wherein the applicator is solid of semi-solid.

15. The system of any one of claims 11-14, wherein the applicator includes methylcellulose.

16. The system of any one of claims 11-15, wherein the applicator is a patch configured to contact the wound.

17. The method of any one of claims 11-16, wherein the HOXA3 is a selected from a nucleic acid sequence and an amino acid sequence.

18. The method of any one of claims 11-17, wherein the HOXA3 is an mRNA coding for HOXA3.

19. The method of any one of claims 11-18, wherein the HOXA3 in comprised on a plasmid.

20. The method of any one of claims 11-19, wherein the HOXA includes a moiety for penetrating a cell.

21. The method of any one of claims 11-20, wherein the HOXA3 is at least 49% identical to a wild-type HOXA3.

22. The method of any one of claim 11-22, wherein the composition includes a lipid nanoparticle comprising the HOXA3.

23. A composition for treating an atherosclerotic lesion, comprising:
HOXA3.

24. A composition for aiding closure of a wound in a subject suffering therefrom, comprising:
HOXA3.

25. The composition of claim 24, wherein the HOXA3 is selected from wild-type or modified.

26. The composition of claim 24 or claim 25, wherein the HOXA3 is a modified HOXA3.

27. The composition of any one of claims 24-26, wherein the HOXA3 is modified to include one or more substitutions selected from L175D, E176K, and E178K.

28. The composition of any one of claims 24-27, comprising a cell penetrating moiety.

29. The composition of any one of claims 24-28, for use in treating one or more of a diabetic wound, aged wound, stump wound, and atherosclerosis.

30. The composition of claim 29, for use in treating diabetic wounds.

31. The composition of claim 29, for use in treating aged wounds.

32. The composition of claim 29, for use in treating stump wounds.

33. The composition of claim 29, for use in treating atherosclerosis.

34. A composition for use in accelerating wound repair in a subject in need thereof, comprising:
a modified HoxA3 molecule; and
a pharmaceutically acceptable carrier.

35. The composition of claim 34, wherein the modified HoxA3 molecule is selected from a protein, a nucleic acid, an mRNA, a plasmid, and a vector.

36. The composition of claim 34 or claim 35, wherein the modified HoxA3 molecule comprises or codes for a protein with one or more substitutions selected from L175D, E176K, and E178K.

37. The composition of any one of claims 34-36, wherein the modified HoxA3 includes a cell penetrating moiety.

38. The composition of any one of claims 34-37, wherein the modified HoxA3 is comprised in a lipid nanoparticle.

39. The composition of any one of claims 34-38, wherein the subject is human.

40. The composition of any one of claims 34-39, wherein the subject is selected from one or more of at least about 60 years of age, diabetic, suffering from immunoaging.

41. The composition of any one of claims 34-40, wherein the composition is comprised in an applicator, and wherein the applicator is solid of semi-solid.

42. The composition of claim 41, wherein the applicator includes methylcellulose.

43. The composition of claim 41, wherein the applicator is a patch configured to contact the wound.

44. Any one of claims 1-43, wherein the HOX is selected from HoxA3, HoxD3, HoxB3, or HoxC3.

45. The method or composition of claim 44, wherein the Hox amino acid sequence includes one or more amino acid substitutions at L175D, E176K, and E178K of HoxA3 or the equivalent position in HoxD3, HoxB3, or HoxC3.
